(19) United States
(12) Patent Application Publication
(10) Pub. No.: US 2025/0197354 A1
(43) Pub. Date: Jun. 19, 2025

(54) SOLID STATE FORMS OF LX9211 AND SALTS THEREOF

(71) Applicant: TEVA PHARMACEUTICALS INTERNATIONAL GMBH, Jona (CH)

(72) Inventors: Dijana Skalec Samec, Jastrebarsko (HR); Klara Karadakic, Zagreb (HR); Tea Stojimanov, Rovinj (HR); Ivana Landeka, Zagreb (HR)

(21) Appl. No.: 18/849,018

(22) PCT Filed: Mar. 23, 2023
(86) PCT No.: PCT/US2023/015992

§ 371 Date: Sep. 20, 2024

Related U.S. Application Data
(60) Provisional application No. 63/406,795, filed on Sep. 15, 2022.
(60) Provisional application No. 63/392,186, filed on Jul. 26, 2022.
(60) Provisional application No. 63/347,746, filed on Jun. 1, 2022.
(60) Provisional application No. 63/334,865, filed on Apr. 26, 2022.
(60) Provisional application No. 63/329,927, filed on Apr. 12, 2022.
(60) Provisional application No. 63/327,868, filed on Apr. 6, 2022.
(60) Provisional application No. 63/322,741, filed on Mar. 23, 2022.

(51) Int. Cl. C07D 213/65

(52) U.S. Cl. CPC C07D 213/65

(57) ABSTRACT

The present disclosure encompasses solid state forms of LX9211, salts and cocrystals thereof, in embodiments processes for preparation thereof, and pharmaceutical compositions thereof.

### FIELD OF THE DISCLOSURE

The present disclosure encompasses solid state forms of LX9211, salts and cocrystals thereof, in embodiments processes for preparation thereof, and pharmaceutical compositions thereof.

### BACKGROUND OF THE DISCLOSURE

LX9211, (S)-1-((2′,6-bis(difluoromethyl)-[2,4′-bipyridin]-5-yl)oxy)-2,4-dimethylpentan-2-amine, has the following chemical structure:

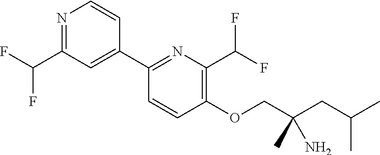 [SMILES: CC(C)C[C@](C)(N)COc1ccc(-c2ccnc(C(F)F)c2)nc1C(F)F]

LX9211 is an investigational oral AAKI inhibitor in clinical development for the treatment of patients with Diabetic Peripheral Neuropathic pain and Post-Herpetic Neuralgia.

The compound is described in International Publication No. WO 2015/153720.

Polymorphism, the occurrence of different crystalline forms, is a property of some molecules and molecular complexes. A single molecule may give rise to a variety of polymorphs having distinct crystal structures and physical properties like melting point, thermal behaviors (e.g., measured by thermogravimetric analysis (“TGA”), or differential scanning calorimetry (“DSC”)), X-ray diffraction (XRD) pattern, infrared absorption fingerprint, and solid state (¹³C) NMR spectrum. One or more of these techniques may be used to distinguish different polymorphic forms of a compound.

Different salts and solid state forms (including solvated forms) of an active pharmaceutical ingredient may possess different properties. Such variations in the properties of different salts and solid state forms and solvates may provide a basis for improving formulation, for example, by facilitating better processing or handling characteristics, changing the dissolution profile in a favorable direction, or improving stability (polymorph as well as chemical stability) and shelf-life. These variations in the properties of different salts and solid state forms may also offer improvements to the final dosage form, for instance, if they serve to improve bioavailability. Different salts and solid state forms and solvates of an active pharmaceutical ingredient may also give rise to a variety of polymorphs or crystalline forms, which may in turn provide additional opportunities to assess variations in the properties and characteristics of a solid active pharmaceutical ingredient.

Discovering new solid state forms and solvates of a pharmaceutical product may yield materials having desirable processing properties, such as ease of handling, ease of processing, storage stability, and ease of purification or as desirable intermediate crystal forms that facilitate conversion to other polymorphic forms. New solid state forms of a pharmaceutically useful compound can also provide an opportunity to improve the performance characteristics of a pharmaceutical product. It enlarges the repertoire of materials that a formulation scientist has available for formulation optimization, for example by providing a product with different properties, including a different crystal habit, higher crystallinity, or polymorphic stability, which may offer better processing or handling characteristics, improved dissolution profile, or improved shelf-life (chemical/physical stability). For at least these reasons, there is a need for additional solid state forms (including solvated forms) of LX9211.

### SUMMARY OF THE DISCLOSURE

The present disclosure provides crystalline polymorphs of LX9211, salts and co-crystals thereof, processes for preparation thereof, and pharmaceutical compositions thereof. Any one of the crystalline polymorphs can be used to prepare other solid state forms of LX9211, LX9211 salts and/or co-crystals thereof and their solid state forms.

The present disclosure also provides uses of said solid state forms of LX9211 or salts and/or cocrystals thereof in the preparation of other solid state forms of LX9211 or salts thereof.

The present disclosure provides crystalline forms of LX9211 or salts and/or cocrystals thereof for use in medicine, including for the treatment of patients with Diabetic Peripheral Neuropathic pain and/or Post-Herpetic Neuralgia.

The present disclosure also encompasses the use of any one or a combination of the crystalline polymorphs of LX9211 or the salts thereof and/or cocrystals thereof of the present disclosure for the preparation of pharmaceutical compositions and/or formulations.

In another aspect, the present disclosure provides pharmaceutical compositions comprising any one of or a combination of the crystalline polymorphs of LX9211 or salts and/or cocrystals thereof according to the present disclosure.

The present disclosure includes processes for preparing the above mentioned pharmaceutical compositions. The processes include combining any one or a combination of the crystalline polymorphs of LX9211 or salts and/or cocrystals thereof with at least one pharmaceutically acceptable excipient.

The crystalline polymorphs of LX9211 as defined herein and the pharmaceutical compositions or formulations of the crystalline polymorph of LX9211 or salts and/or cocrystals thereof may be used as medicaments, such as for the treatment of patients with Diabetic Peripheral Neuropathic pain and/or Post-Herpetic Neuralgia.

The present disclosure also provides methods for the treatment of patients with Diabetic Peripheral Neuropathic pain and/or Post-Herpetic Neuralgia by administering a therapeutically effective amount of any one or a combination of the crystalline polymorphs of LX9211 or salts thereof of the present disclosure, or at least one of the above pharmaceutical compositions, to a subject suffering from Diabetic Peripheral Neuropathic pain and/or Post-Herpetic Neuralgia or otherwise in need of the treatment.

The present disclosure also provides uses of crystalline polymorphs of LX9211 or salts and/or cocrystals thereof of the present disclosure, or at least one of the above pharmaceutical compositions, for the manufacture of medicaments for treating e.g., patients with Diabetic Peripheral Neuropathic pain and/or Post-Herpetic Neuralgia.

### BRIEF DESCRIPTION OF THE DRAWINGS

FIG. 1 shows a characteristic X-ray powder diffraction pattern (XRPD) of LX9211 Form A (the peak at 28.50 degrees two-theta corresponds to Si).

FIG. 2 shows the X-ray powder diffraction pattern (XRPD) of LX9211 obtained according to example 2, procedure A.

FIG. 2A shows the X-ray powder diffraction pattern (XRPD) of LX9211 form B obtained according to example 2, procedure E.

FIG. 3 shows the X-ray powder diffraction pattern (XRPD) of LX9211 dihydrogen phosphate form S1.

FIG. 3A shows the X-ray powder diffraction pattern (XRPD) of LX9211 dihydrogen phosphate form S1 obtained according to example 3, procedure B.

FIG. 3B shows the crystal structure of LX9211 dihydrogen phosphate form S1.

FIG. 3C shows the calculated powder diffraction pattern of LX9211 dihydrogen phosphate form S1 (lower trace) compared to the measured diffractogram (upper trace).

FIG. 4 shows the X-ray powder diffraction pattern (XRPD) of LX9211 dihydrogen phosphate form S3.

FIG. 5 shows the X-ray powder diffraction pattern (XRPD) of LX9211 Tosylate form T1.

FIG. 6 shows the X-ray powder diffraction pattern (XRPD) of LX9211 Tosylate form T2.

FIG. 7 shows the X-ray powder diffraction pattern (XRPD) of LX9211 Camsylate form C1.

FIG. 8 shows the X-ray powder diffraction pattern (XRPD) of LX9211 Camsylate form C2.

FIG. 9 shows the X-ray powder diffraction pattern (XRPD) of form S5 of LX9211: Phosphoric acid (or LX9211 hemihydrogen phosphate).

FIG. 9A shows the crystal structure of LX9211: phosphoric acid (or LX9211 hemihydrogen phosphate) form S5.

FIG. 9B shows the calculated powder diffraction pattern of LX9211 hemi-hydrogen phosphate form S5 (lower trace) compared to the measured diffractogram (upper trace).

FIG. 10 shows the X-ray powder diffraction pattern (XRPD) of LX9211 Succinate form J2.

FIG. 11 shows the X-ray powder diffraction pattern (XRPD) of LX9211 Fumarate form F3.

FIG. 12 shows the X-ray powder diffraction pattern (XRPD) of LX9211 Camsylate form K1.

FIG. 13 shows the X-ray powder diffraction pattern (XRPD) of LX9211 Citrate form L1.

FIG. 14 shows the X-ray powder diffraction pattern (XRPD) of LX9211 Citrate form L2.

FIG. 15 shows the X-ray powder diffraction pattern (XRPD) of LX9211 Oxalate form O5 (Si peak at 28.48° 2θ).

FIG. 16 shows the X-ray powder diffraction pattern (XRPD) of LX9211 Tartrate form V2.

FIG. 17 shows the X-ray powder diffraction pattern (XRPD) of form U1 of LX9211 dihydrogen phosphate: Urea (Si peak at 28.46° 2θ).

FIG. 18 shows the X-ray powder diffraction pattern (XRPD) of form V3 of LX9211 dihydrogen phosphate: L-(+)-Tartaric acid (or LX9211: phosphoric acid: L-(+)-Tartaric acid).

FIG. 19 shows the X-ray powder diffraction pattern (XRPD) of form O4 of LX9211 dihydrogen phosphate: Oxalic acid (or LX9211 phosphoric acid: Oxalic acid).

FIG. 20 shows the X-ray powder diffraction pattern (XRPD) of form C of LX9211.

FIG. 21 shows the X-ray powder diffraction pattern (XRPD) of LX9211 dihydrogen phosphate form S4.

FIG. 22 shows the X-ray powder diffraction pattern (XRPD) of LX9211 oxalate form O2.

FIG. 23 shows the X-ray powder diffraction pattern (XRPD) of LX9211 tartrate form V5.

FIG. 24 shows the solid state ¹³C-NMR of LX9211 dihydrogen phosphate form S1 (200-0 ppm).

FIG. 25 shows the solid state ¹³C-NMR of LX9211 dihydrogen phosphate form S3 (200-0 ppm).

FIG. 26 shows the solid state ¹³C-NMR of LX9211: phosphoric acid (or LX9211 hemihydrogen phosphate) form S5 (200-0 ppm).

FIG. 27 shows the solid state ¹³C-NMR of form U1 of LX9211 dihydrogen phosphate: urea (or LX9211 phosphoric acid: Urea) (200-0 ppm).

### DETAILED DESCRIPTION OF THE DISCLOSURE

The present disclosure encompasses a crystalline form of LX9211, processes for preparation thereof, and pharmaceutical compositions thereof.

Solid state properties of LX9211 and crystalline polymorphs thereof can be influenced by controlling the conditions under which LX9211 and crystalline polymorphs thereof are obtained in solid form.

The solid state forms of LX9211 (e.g. LX9211, LX9211 salts, or cocrystals) as described in any aspect or embodiment of the present disclosure may be polymorphically pure, or substantially free of any other solid state (or polymorphic) forms.

A solid state form (or polymorph) may be referred to herein as polymorphically pure or as substantially free of any other solid state (or polymorphic) forms. As used herein in this context, the expression “substantially free of any other forms” will be understood to mean that the solid state form contains about 20% (w/w) or less, about 10% (w/w) or less, about 5% (w/w) or less, about 2% (w/w) or less, about 1% (w/w) or less, or about 0% of any other forms of the subject compound as measured, for example, by XRPD. For example, polymorphically pure LX9211 dihydrogen phosphate form S1 means that the solid state form is substantially free of other solid state forms of LX9211 dihydrogen phosphate. Thus, a crystalline polymorph of LX9211, salt or cocrystal described herein as substantially free of any other solid state forms would be understood to contain greater than about 80% (w/w), greater than about 90% (w/w), greater than about 95% (w/w), greater than about 98% (w/w), greater than about 99% (w/w), or about 100% of the subject crystalline polymorph of LX9211, salt or cocrystal. In some embodiments of the disclosure, the described crystalline polymorph of LX9211, salt or cocrystal may contain from about 1% to about 20% (w/w), from about 5% to about 20% (w/w), or from about 5% to about 10% (w/w) of one or more other crystalline polymorph of the same LX9211.

Depending on which other crystalline polymorphs a comparison is made, the crystalline polymorphs of LX9211, LX9211 salts, such as LX9211 dihydrogen phosphate, LX9211 hemihydrogen phosphate, LX 9211 camsylate or LX9211 cocrystals such as LX9211 dihydrogen phosphate: urea of the present disclosure may have advantageous properties selected from at least one of the following: chemical purity, flowability, solubility, dissolution rate, morphology or crystal habit, stability, such as chemical stability as well as thermal and mechanical stability with respect to polymorphic conversion, stability towards dehydration and/or storage stability, low content of residual solvent, a lower degree of hygroscopicity, flowability, and advantageous processing and handling characteristics such as compressibility and bulk density. Particularly, a crystalline form of LX9211, LX9211 dihydrogen phosphate, LX9211 hemi-hydrogen phosphate, LX9211 camsylate and LX9211 dihydrogen phosphate: Urea co-crystal, as described in any aspect or embodiment of the present disclosure, may be stable, for example to conditions of high relative humidity, and/or may be thermally stable. Crystalline form S1 of LX9211 dihydrogen phosphate and crystalline form S5 of LX9211 hemi-hydrogen phosphate may be especially stable to conditions of high humidity. Crystalline form S3 of LX9211 dihydrogen phosphate may be especially thermally stable. Crystalline form K1 of LX9211 camsylate may be especially stable to conditions of high relative humidity and/or may be thermally stable. Crystalline form U1 of LX9211 dihydrogen phosphate: Urea may exhibit improved solubility.

A solid state form, such as a crystal form or an amorphous form, may be referred to herein as being characterized by graphical data “as depicted in” or “as substantially depicted in” a Figure. Such data include, for example, powder X-ray diffractograms and solid state NMR spectra. As is well-known in the art, the graphical data potentially provides additional technical information to further define the respective solid state form (a so-called “fingerprint”) which cannot necessarily be described by reference to numerical values or peak positions alone. In any event, the skilled person will understand that such graphical representations of data may be subject to small variations, e.g., in peak relative intensities and peak positions due to certain factors such as, but not limited to, variations in instrument response and variations in sample concentration and purity, which are well known to the skilled person. Nonetheless, the skilled person would readily be capable of comparing the graphical data in the Figures herein with graphical data generated for an unknown crystal form and confirm whether the two sets of graphical data are characterizing the same crystal form or two different crystal forms. A crystal form of LX9211 (e.g. LX9211, LX9211 salts, or cocrystals) referred to herein as being characterized by graphical data “as depicted in” or “as substantially depicted in” a Figure will thus be understood to include any crystal forms of LX9211 characterized with the graphical data having such small variations, as are well known to the skilled person, in comparison with the Figure.

As used herein, and unless stated otherwise, the term “anhydrous” in relation to crystalline forms of LX9211 (e.g. LX9211, LX9211 salts, or cocrystals) relates to a crystalline form of LX9211 which does not include any crystalline water (or other solvents) in a defined, stoichiometric amount within the crystal. Moreover, an “anhydrous” form would generally not contain more than 1% (w/w), of either water or organic solvents as measured for example by TGA.

The term “solvate,” as used herein and unless indicated otherwise, refers to a crystal form that incorporates a solvent in the crystal structure. When the solvent is water, the solvate is often referred to as a “hydrate.” The solvent in a solvate may be present in either a stoichiometric or in a non-stoichiometric amount.

As used herein, unless stated otherwise, unit cell information was obtained by solving the crystal structure.

“Co-Crystal” or “Co-crystal” as used herein is defined as a crystalline material including two or more molecules in the same crystalline lattice and associated by non-ionic and non-covalent bonds. In some embodiments, the co-crystal includes two molecules which are in natural state.

As used herein, crystalline LX9211 dihydrogen phosphate: Urea (or crystalline LX9211 phosphoric acid: Urea) is a distinct molecular species. Crystalline LX9211 dihydrogen phosphate: Urea (or LX9211 phosphoric acid: Urea) may be a co-crystal of LX9211 dihydrogen phosphate and Urea. Alternatively, crystalline LX9211 dihydrogen phosphate: Urea (or LX9211 phosphoric acid: Urea) may be a salt, preferably crystalline LX9211 dihydrogen phosphate: Urea (or LX9211 phosphoric acid: Urea) may be a co-crystal of LX9211 dihydrogen phosphate and Urea.

As used herein, crystalline LX9211 dihydrogen phosphate: L-(+)-Tartaric acid (or LX9211 phosphoric acid: L-(+)-Tartaric acid) is a distinct molecular species. Crystalline LX9211 dihydrogen phosphate: L-(+)-Tartaric acid (or LX9211 phosphoric acid: L-(+)-Tartaric acid) may be a co-crystal of LX9211 dihydrogen phosphate and L-(+)-Tartaric acid. Alternatively, crystalline LX9211 dihydrogen phosphate: L-(+)-Tartaric acid (or LX9211 phosphoric acid: L-(+)-Tartaric acid) may be a salt.

As used herein, crystalline LX9211 dihydrogen phosphate: Oxalic acid (or LX9211 phosphoric acid: Oxalic acid) is a distinct molecular species. Crystalline LX9211 dihydrogen phosphate: Oxalic acid (or LX9211 phosphoric acid: Oxalic acid) may be a co-crystal of LX9211 dihydrogen phosphate and Oxalic acid. Alternatively, crystalline LX9211 dihydrogen phosphate: Oxalic acid (or LX9211 phosphoric acid: Oxalic acid) may be a salt.

As used herein, the term “isolated” in reference to crystalline polymorph of LX9211 of the present disclosure corresponds to a crystalline polymorph of LX9211 that is physically separated from the reaction mixture in which it is formed.

As used herein, unless stated otherwise, the XRPD measurements are taken using copper K α radiation wavelength 1.54184 Å. XRPD peaks reported herein are optionally measured using Cuk α radiation, λ=1.54184 Å, typically at a temperature of 25±3° C.

As used herein, unless stated otherwise, the ¹³C CP/MAS spectra employing cross-polarization were acquired using the standard cross-polarization pulse scheme at spinning frequency 18 kHz. on 700 MHZ NMR instrument, or at spinning frequency 11 kHz on 500 MHZ NMR instrument. In particular, ¹³C solid state NMR for Forms S1, S3 and S5 were measured at 700 MHz at a spinning frequency of 18 kHz. The ¹³C solid state NMR for Form U1 was measured at 500 MHz at a spinning frequency of 11 kHz.

A thing, e.g., a reaction mixture, may be characterized herein as being at, or allowed to come to “room temperature” or “ambient temperature,” often abbreviated as “RT.” This means that the temperature of the thing is close to, or the same as, that of the space, e.g., the room or fume hood, in which the thing is located. Typically, room temperature is from about 20° C. to about 30° C., or about 22° C. to about 27° C., or about 25° C.

The amount of solvent employed in a chemical process, e.g., a reaction or crystallization, may be referred to herein as a number of “volumes” or “vol” or “V.” For example, a material may be referred to as being suspended in 10 volumes (or 10 vol or 10V) of a solvent. In this context, this expression would be understood to mean milliliters of the solvent per gram of the material being suspended, such that suspending a 5 grams of a material in 10 volumes of a solvent means that the solvent is used in an amount of 10 milliliters of the solvent per gram of the material that is being suspended or, in this example, 50 mL of the solvent. In another context, the term “v/v” may be used to indicate the number of volumes of a solvent that are added to a liquid mixture based on the volume of that mixture. For example, adding solvent X (1.5 v/v) to a 100 ml reaction mixture would indicate that 150 mL of solvent X was added.

A process or step may be referred to herein as being carried out “overnight.” This refers to a time interval, e.g., for the process or step, that spans the time during the night, when that process or step may not be actively observed. This time interval is from about 8 to about 20 hours, or about 10-18 hours, in some cases about 16 hours.

As used herein, the term “reduced pressure” refers to a pressure that is less than atmospheric pressure. For example, reduced pressure is about 10 mbar to about 50 mbar.

As used herein and unless indicated otherwise, the term “ambient conditions” refer to atmospheric pressure and a temperature of 22-24° C.

The solid state forms of LX9211 (e.g. LX9211, LX9211 salts, or cocrystals) as described in any aspect or embodiment of the present disclosure may be chemically pure, or substantially free of any other compounds.

A compound may be referred to herein as chemically pure or purified compound or as substantially free of any other compounds. As used herein, the terms “chemically pure” or “purified” or “substantially free of any other compounds” refer to a compound that is substantially free of any impurities including enantiomers of the subject compound, diastereomers or other isomers. A chemically pure or purified compound or a compound that is substantially free of any other compound will be understood to mean that it contains about 10% (w/w) or less, about 5% (w/w) or less, about 4% (w/w) or less, about 3% (w/w) or less, about 2% (w/w) or less, about 1.5% (w/w) or less, about 1% (w/w) or less, about 0.8% (w/w) or less, about 0.6% (w/w) or less, about 0.4% (w/w) or less, about 0.2% (w/w) or less, about 0.1% (w/w) or less, or about 0% of any other compound as measured, for example, by HPLC. Alternatively, A chemically pure or purified compound or a compound that is substantially free of any other compound will be understood to mean that it contains about 10% area percent or less, about 5% area percent or less, about 4% area percent or less, about 3% area percent or less, about 2% area percent or less, about 1.5% area percent or less, about 1% area percent or less, about 0.8% area percent or less, about 0.6% area percent or less, about 0.4% area percent or less, about 0.2% area percent or less, about 0.1% area percent or less, or about 0% of any other compound as measured by HPLC. Thus, pure or purified LX9211, salts or co-crystal thereof or LX9211 intermediate described herein as substantially free of any compounds would be understood to contain greater than about 90% (w/w), greater than about 95% (w/w), greater than about 96% (w/w), greater than about 97% (w/w), greater than about 98% (w/w), greater than about 98.5% (w/w), greater than about 99% (w/w), greater than about 99.2% (w/w), greater than about 99.4% (w/w), greater than about 99.6% (w/w), greater than about 99.8% (w/w), greater than about 99.9% (w/w), or about 100% of the subject LX9211, salts or co-crystal thereof or LX9211 intermediate. Alternatively, pure or purified LX9211, salts or co-crystal thereof or LX9211 intermediate described herein as substantially free of any compounds would be understood to contain greater than about 90% area percent, greater than about 95% area percent, greater than about 96% area percent, greater than about 97% area percent, greater than about 98% area percent, greater than about 98.5% area percent, greater than about 99% area percent, greater than about 99.2% area percent, greater than about 99.4% area percent, greater than about 99.6% area percent, greater than about 99.8% area percent, greater than about 99.9% area percent, or about 100% of the subject LX9211 salts or co-crystal thereof or LX9211 intermediate.

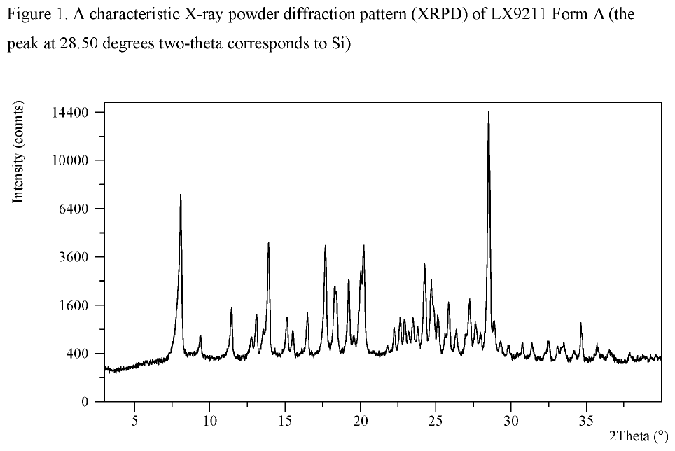

The present disclosure includes a crystalline polymorph LX9211 designated Form A. The crystalline Form A of LX9211 may be characterized by data selected from one or more of the following: an X-ray powder diffraction pattern substantially as depicted in FIG. 1; an X-ray powder diffraction pattern having peaks at 8.0, 13.9 and 17.6 degrees 2-theta±0.2 degrees 2-theta; and combinations of these data.

Crystalline Form A of LX9211 may be further characterized by an X-ray powder diffraction pattern having peaks at 8.0, 13.9 and 17.6 degrees 2-theta±0.2 degrees 2-theta, and also having any one or both additional peaks selected from 19.2 and 24.2 degrees 2-theta±0.2 degrees 2-theta.

Crystalline Form A of LX9211 may be alternatively characterized by an X-ray powder diffraction pattern having peaks at 8.0, 13.9, 17.6, 19.2 and 24.2 degrees 2-theta±0.2 degrees 2-theta.

Crystalline Form A of LX9211 may be further characterized by an X-ray powder diffraction pattern having peaks at 8.0, 13.9, 17.6, 19.2 and 24.2 degrees 2-theta±0.2 degrees 2-theta, and also having any one, two, three, four or five additional peaks selected from 9.3, 11.4, 15.1, 15.5 and 16.4 degrees 2-theta±0.2 degrees 2-theta.

Crystalline Form A of LX9211 may be alternatively characterized by an X-ray powder diffraction pattern having peaks at 8.0, 9.3, 11.4, 13.9, 15.1, 15.5, 16.4, 17.6, 19.2 and 24.2 degrees 2-theta±0.2 degrees 2-theta.

In any aspect or embodiment of the present disclosure, crystalline Form A of LX9211 is isolated. Particularly, crystalline Form A of LX9211 according to any aspect or embodiment of the disclosure may be isolated.

In any aspect or embodiment crystalline Form A of LX9211 may be chemically pure.

In any aspect or embodiment crystalline Form A of LX9211 may be polymorphically pure.

In any aspect or embodiment of the present disclosure, crystalline Form A of LX9211 may be anhydrous.

Crystalline Form A of LX9211 may be characterized by each of the above characteristics alone/or by all possible combinations, e.g., an XRPD pattern having peaks at 8.0, 13.9, 17.6, 19.2 and 24.2 degrees 2-theta±0.2 degrees 2-theta; an XRPD pattern as depicted in FIG. 1, and combinations thereof.

Form A of LX9211 may be prepared by crystallizing LX9211 in a polar organic solvent. The polar organic solvent may comprise one or more solvents. Suitable solvents may include but are not limited to alcohols, esters, ethers, ketones and halogenated hydrocarbons. The alcohols are preferably a C₁ to C₆ alcohol, more preferably a C₁₋₄ alcohol, and particularly methanol or ethanol. The ester solvent is preferably a C3 to C₈ ester, more preferably a C₃ to C₆ ester, and particularly ethyl acetate. The ketone is preferably a C₃ to C₈ ketone, more preferably a C₃ to C₆ ketone, and particularly acetone. The ether is preferably a C₄ to C₈ ether, more preferably a C₄-C₆ ether, particularly wherein the ether is diethyl ether or diisopropyl ether. The halogenated hydrocarbon is preferably a C₁ to C₆ alkane which is substituted with 1-6 halo groups, preferably chloro or fluoro, and more preferably the halogenated hydrocarbon is a C₁ to C; hydrocarbon which is substituted with 1-4 chloro groups or a C₁ to C₂ hydrocarbon which is substituted with 1-3 chloro groups, and more particularly chloroform or dichloromethane. In a preferred process, Form A of LX9211 may be prepared by crystallising LX9211 in a solvent selected from the group consisting of ethanol, methanol, ethyl acetate, diethyl ether, diisopropyl ether, acetone, chloroform and dichloromethane. The solvent is used in an amount to at least dissolve the LX9211, and is preferably in the range of: about 2 ml to about 100 ml, about 2 ml to about 80 ml, about 2 ml to about 60 ml, about 4 ml to about 50 ml, or about 6 ml to about 40 ml, per gram of LX9211. The process may comprise obtaining a solution of LX9211 in the polar organic solvent and crystallizing form A, preferably by evaporation of the solvent. The evaporation is preferably carried out slowly, for example by evaporation of the solvent through one or more small apertures in a covered receptacle. For example, the evaporation may be conducted over a period of: about 1 to about 7 days, about 1 to about 3 days, about 1 to about 3 days, or about 2 days. The evaporation may be conducted at a temperature of about 0° C. to about 30° C., about 0° C. to about 25° C., about 0° C. to about 20° C., about 0° C. to about 15° C., about 2° C. to about 12° C., or about 5° C. to about 10° C.

The present disclosure further encompasses a crystalline product obtainable by any of the above processes.

The process may further comprise combining Form A of LX9211 with at least one pharmaceutically acceptable excipient to form a pharmaceutical composition.

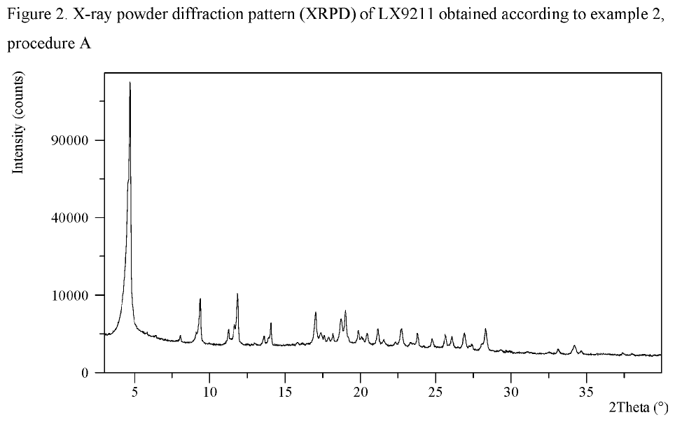

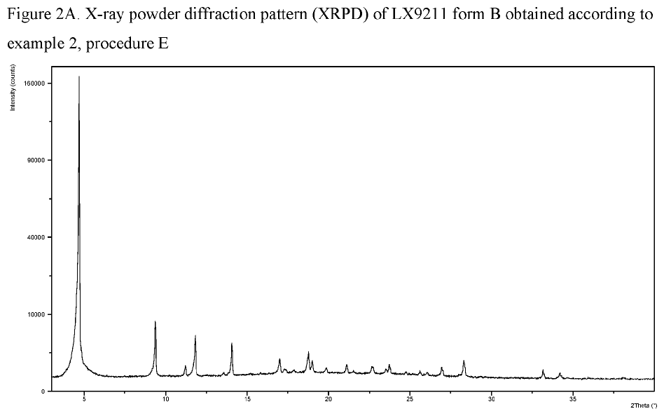

The present disclosure includes a crystalline polymorph LX9211 designated Form B. The crystalline Form B of LX9211 may be characterized by data selected from one or more of the following: an X-ray powder diffraction pattern substantially as depicted in FIG. 2A; an X-ray powder diffraction pattern having peaks at 4.6, 11.9 and 17.0 degrees 2-theta±0.2 degrees 2-theta; and combinations of these data.

Crystalline Form B of LX9211 may be further characterized by an X-ray powder diffraction pattern having peaks at 4.6, 11.9 and 17.0 degrees 2-theta±0.2 degrees 2-theta, and also having any one or both additional peaks selected from 14.1 and 21.2 degrees 2-theta±0.2 degrees 2-theta.

Crystalline Form B of LX9211 may be alternatively characterized by an X-ray powder diffraction pattern having peaks at 4.6, 11.9, 14.1, 17.0 and 21.2 degrees 2-theta±0.2 degrees 2-theta.

Crystalline Form B of LX9211 may be further characterized by an X-ray powder diffraction pattern having peaks at 4.6, 11.9, 14.1, 17.0 and 21.2 degrees 2-theta±0.2 degrees 2-theta, and also having any one, two, three, four or five additional peaks selected from 11.2, 17.4, 18.7, 19.0 and 20.4 degrees 2-theta±0.2 degrees 2-theta.

Crystalline Form B of LX9211 may be alternatively characterized by an X-ray powder diffraction pattern having peaks at 4.6, 11.2, 11.9, 14.1, 17.0, 17.4, 18.7, 19.0, 20.4 and 21.2 degrees 2-theta±0.2 degrees 2-theta.

In any aspect or embodiment of the present disclosure, crystalline Form B of LX9211 is isolated. Particularly, crystalline Form B of LX9211 according to any aspect or embodiment of the disclosure may be isolated.

In any aspect or embodiment crystalline Form B of LX9211 may be chemically pure.

In any aspect or embodiment crystalline Form B of LX9211 may be polymorphically pure.

In any aspect or embodiment of the present disclosure, crystalline Form B of LX9211 may be anhydrous.

Crystalline Form B of LX9211 may be characterized by each of the above characteristics alone/or by all possible combinations, e.g., an XRPD pattern having peaks at 4.6, 11.9, 14.1, 17.0 and 21.2 degrees 2-theta±0.2 degrees 2-theta; an XRPD pattern as depicted in FIG. 2A, and combinations thereof.

Form B of LX9211 may be prepared by slurrying LX9211 in a mixture of an alcohol and water. The starting form of LX9211 may be any form of LX9211 but is preferably form A as described in any aspect or embodiment of the present disclosure. Preferably, the slurrying comprises stirring LX9211 in a mixture of alcohol and water. The alcohol is preferably a C₁ to C₈ alcohol, more preferably a C₁-C₆ alcohol, most preferably a C₁₋₄ alcohol or a C₁₋₃ alcohol, and particularly methanol or isopropanol. The mixture of alcohol and water may comprise the alcohol in an amount of: about 2% to about 50%, about 5% to about 40%, about 8% to about 30%, about 10% to about 20%, about 12% to about 18%, or about 15%, by volume. In any aspect or embodiment of the process, the stirring may be carried out at a temperature of about-5° C. to about 30° C., about-2° C. to about 25° C., about 0° C. to about 20° C., about 0° C. to about 15° C. or particularly about 0° C. to about 10° C. The stirring may be carried out for any suitable time to prepare Form B of LX9211. Preferably, the stirring may be for a period of: about 1 hour to about 9 days, about 2 hours to about 7 days, or about 2.5 hours to about 6 days, about 3 days to about 6 days or about 4 days to about 6 days. The product may be isolated by any suitable procedure, such as decantation, centrifugation or filtration, preferably by filtration. The process may further comprise combining the Form B of LX9211 with at least one pharmaceutically acceptable excipient to form a pharmaceutical composition.

Form B may also be obtained by exposing form A to conditions of high relative humidity, particularly at about 98% to about 100%, or about 100% RH. Preferably, the temperature is: about 20° C. to about 50° C., about 30° C. to about 45° C., about 35° C. to about 45° C., or about 40° C. Particularly, Form B may be prepared by exposing Form A to 98%-100% RH, or particularly about 100% RH, at a temperature of about 20° C. to about 50° C., preferably at a temperature of about 40° C. The exposure may be for a sufficient period of time to prepare Form B, preferably about 7 days to about 2 months, about 14 days to about 40 days, or about 21 days to about 31 days, or about 1 month.

The present disclosure further encompasses a crystalline product obtainable by any of the above processes. The process may further comprise combining the Form B of LX9211 with at least one pharmaceutically acceptable excipient to form a pharmaceutical composition.

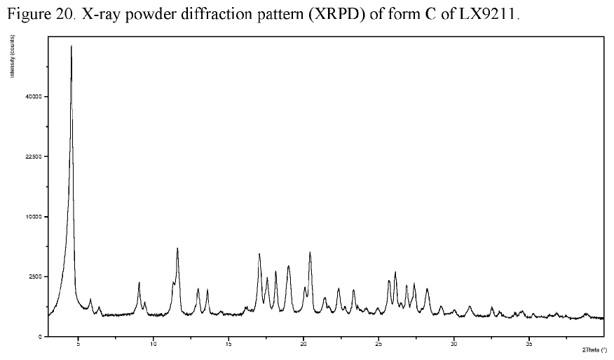

The present disclosure includes a crystalline polymorph LX9211 designated Form C. The crystalline Form C of LX9211 may be characterized by data selected from one or more of the following: an X-ray powder diffraction pattern substantially as depicted in FIG. 20; an X-ray powder diffraction pattern having peaks at 9.1, 13.0 and 18.2 degrees 2-theta±0.2 degrees 2-theta; and combinations of these data.

Crystalline Form C of LX9211 may be further characterized by an X-ray powder diffraction pattern having peaks at 9.1, 13.0 and 18.2 degrees 2-theta±0.2 degrees 2-theta, and also having any one or both additional peaks selected from 11.6 and 22.3 degrees 2-theta±0.2 degrees 2-theta.

Crystalline Form C of LX9211 may be alternatively characterized by an X-ray powder diffraction pattern having peaks at 9.1, 11.6, 13.0, 18.2 and 22.3 degrees 2-theta±0.2 degrees 2-theta.

Crystalline Form C of LX9211 may be further characterized by an X-ray powder diffraction pattern having peaks at 9.1, 11.6, 13.0, 18.2 and 22.3 degrees 2-theta±0.2 degrees 2-theta, and also having any one, two, three, four or five additional peaks selected from 5.8, 17.6, 20.4, 21.4 and 23.3 degrees 2-theta±0.2 degrees 2-theta.

Crystalline Form C of LX9211 may be alternatively characterized by an X-ray powder diffraction pattern having peaks at 5.8, 9.1, 11.6, 13.0, 17.6, 18.2, 20.4, 21.4, 22.3 and 23.3 degrees 2-theta±0.2 degrees 2-theta.

In any aspect or embodiment of the present disclosure, crystalline Form C of LX9211 is isolated. Particularly, crystalline Form C of LX9211 according to any aspect or embodiment of the disclosure may be isolated.

In any aspect or embodiment crystalline Form C of LX9211 may be chemically pure.

In any aspect or embodiment crystalline Form C of LX9211 may be polymorphically pure.

In any aspect or embodiment of the present disclosure, crystalline Form C of LX9211 may be anhydrous.

Crystalline Form C of LX9211 may be characterized by each of the above characteristics alone/or by all possible combinations, e.g., an XRPD pattern having peaks at 9.1, 11.6, 13.0, 18.2 and 22.3 degrees 2-theta±0.2 degrees 2-theta; an XRPD pattern as depicted in FIG. 20, and combinations thereof.

Form C of LX9211 may be prepared by slurrying LX9211 in water. The starting form of LX9211 may be any form of LX9211 but is preferably form A as described in any aspect or embodiment of the present disclosure. The water may be used in an amount of: about 30 ml to about 200 ml, about 40 ml to about 150 ml, about 60 ml to about 140 ml, about 80 ml to about 120 ml, about 90 ml to about 110 ml, or about 100 ml, per gram of LX9211. Preferably, the slurrying comprises stirring LX9211 in water. In any aspect or embodiment of the process, the stirring may be carried out at a temperature of about 25° C. to about 50° C., but is preferably at about 35° C. to about 40° C., or about 37° C. The stirring may be carried out for any suitable time to prepare Form C of LX9211. Preferably, the stirring may be for a period of: about 1 hour to about 72 hours, about 2 hours to about 48 hours, or about 24 hours. Optionally, the mixture can be cooled, preferably passively, to room temperature. The product may be isolated by any suitable procedure, such as decantation, centrifugation or filtration, preferably by filtration.

The present disclosure further encompasses a crystalline product obtainable by any of the above processes. The process may further comprise combining the Form C of LX9211 with at least one pharmaceutically acceptable excipient to form a pharmaceutical composition.

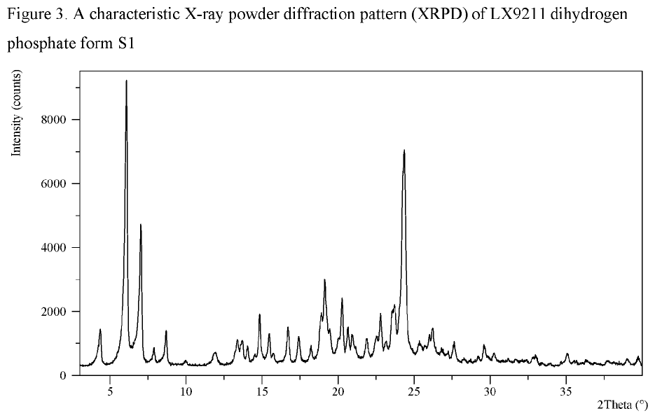

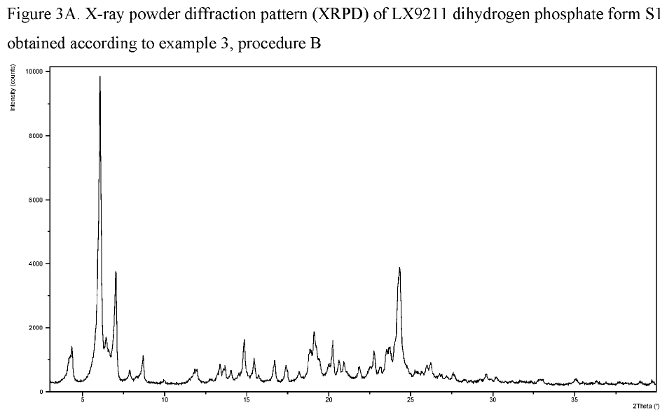

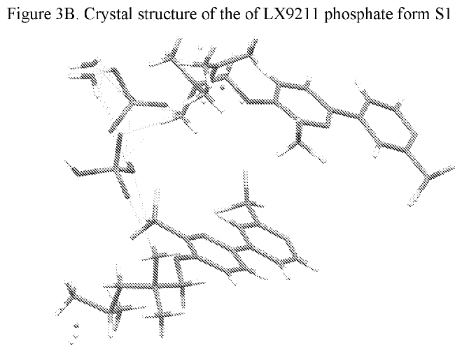

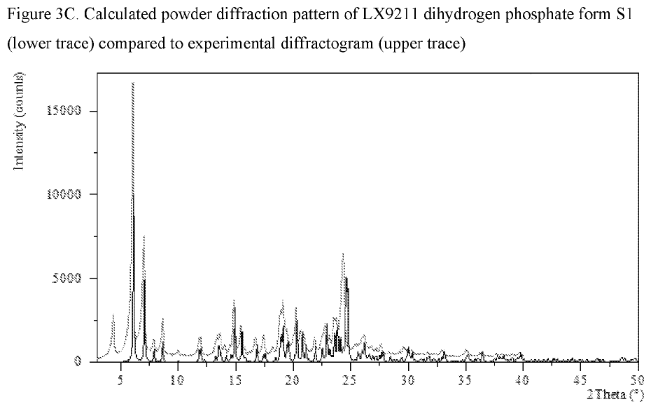

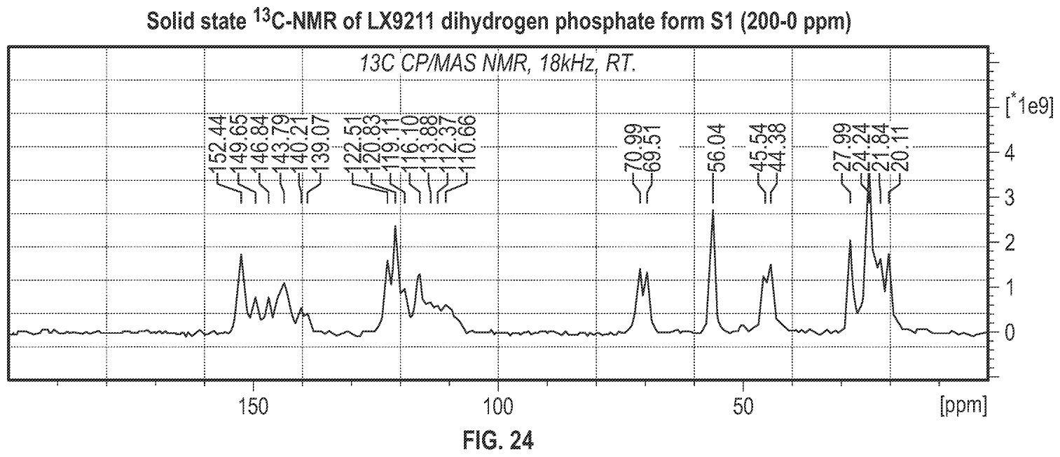

The present disclosure includes a crystalline polymorph LX9211 dihydrogen phosphate designated Form S1. The crystalline Form S1 of LX9211 dihydrogen phosphate may be characterized by data selected from one or more of the following: an X-ray powder diffraction pattern substantially as depicted in FIG. 3 or FIG. 3A; an X-ray powder diffraction pattern having peaks at 7.0, 14.0 and 16.7 degrees 2-theta±0.2 degrees 2-theta; a solid state ¹³C NMR spectrum having characteristic peaks at 152.4, 120.8, 56.0 and 24.2 ppm±0.2 ppm; A solid state ¹³C NMR spectrum having the following chemical shift absolute differences from reference peak at 20.1 ppm±0.2 ppm: 132.3, 100.7, 35.9 and 4.1 ppm±0.1 ppm; a solid state ¹³C NMR spectrum substantially as depicted in FIG. 24; and combinations of these data.

Crystalline Form S1 of LX9211 dihydrogen phosphate may be further characterized by an X-ray powder diffraction pattern having peaks at 7.0, 14.0 and 16.7 degrees 2-theta±0.2 degrees 2-theta, and also having any one or both additional peaks selected from 20.2 and 21.9 degrees 2-theta±0.2 degrees 2-theta.

The crystalline Form S1 of LX9211 dihydrogen phosphate may be alternatively characterized by an X-ray powder diffraction pattern having peaks at 7.0, 14.0, 16.7, 20.2 and 21.9±0.2 degrees 2-theta; an XRPD pattern as depicted in FIG. 3 and combinations of these data.

Crystalline Form S1 of LX9211 dihydrogen phosphate may be further characterized by an X-ray powder diffraction pattern having peaks at 7.0, 14.0, 16.7, 20.2 and 21.9±0.2 degrees 2-theta±0.2 degrees 2-theta, and also having any one, two, three, four or five additional peaks selected from 4.4, 8.7, 10.0, 17.5 and 18.2±0.2 degrees 2-theta±0.2 degrees 2-theta.

Crystalline Form S1 of LX9211 dihydrogen phosphate may be alternatively characterized by an X-ray powder diffraction pattern having peaks at 4.4, 7.0, 8.7, 10.0, 14.0, 16.7, 17.5, 18.2, 20.2 and 21.9 degrees 2-theta±0.2 degrees 2-theta.

In any aspect or embodiment of the present disclosure, crystalline Form S1 of LX9211 dihydrogen phosphate is isolated. Particularly, crystalline Form S1 of LX9211 dihydrogen phosphate according to any aspect or embodiment of the disclosure may be isolated.

In any aspect or embodiment crystalline Form S1 of LX9211 dihydrogen phosphate may be chemically pure.

In any aspect or embodiment crystalline Form S1 of LX9211 dihydrogen phosphate may be polymorphically pure.

In any aspect or embodiment of the present disclosure, crystalline form S1 of LX9211 dihydrogen phosphate may be a hydrate, preferably a monohydrate. In any embodiment, crystalline Form S1 of LX9211 dihydrogen phosphate may contain from about 2% to about 4% of water, preferably about 3%, particularly about 2.8% of water, as measured by TGA.

Alternatively the present disclosure provides form SI of LX9211 dihydrogen phosphate can be characterized by the following unit cell data:

| cell length a | 23.67 | Å |
|---|---|---|
| cell length b | 7.81 | Å |
| cell length c | 26.45 | Å |
| cell angle alpha | 90° | |
| cell angle_beta | 109.6° | |
| cell angle gamma | 90° | |
| cell volume | 4604.48 | A³ |

symmetry cell setting monoclinic
symmetry space group name_I2

Cell data is preferably measured at 170K. Alternatively, form S1 of LX9211 dihydrogen phosphate as defined in any aspect or embodiment herein may be additionally characterized by the above unit cell data.

Crystalline Form S1 of LX9211 dihydrogen phosphate may be characterized by each of the above characteristics alone/or by all possible combinations, e.g., an XRPD pattern having peaks at 7.0, 14.0, 16.7, 20.2 and 21.9 degrees 2-theta±0.2 degrees 2-theta; an XRPD pattern as depicted in FIG. 3 or FIG. 3A, and combinations thereof.

Crystalline Form S1 of LX9211 dihydrogen phosphate according to any aspect or embodiment of the present disclosure may be advantageously stable, for example to conditions of high relative humidity. For example, Form SI may show no polymorphic changes when exposed to 100% RH (e.g. at room temperature), e.g. for up to 1 month.

Form S1 of LX9211 dihydrogen phosphate as described in any aspect or embodiment of the present disclosure may be prepared by crystallizing LX9211 dihydrogen phosphate in an alcohol or a mixture of an alcohol and water. The mixture of LX9211 dihydrogen phosphate in an alcohol or a mixture of an alcohol and water may be prepared by obtaining a solution of LX9211 dihydrogen phosphate in the alcohol (preferably 2-propanol), and optionally adding water. The solution of LX9211 dihydrogen phosphate in the alcohol may be obtained by dissolving LX9211 in the alcohol and adding phosphoric acid (preferably concentrated phosphoric acid, particularly 85% phosphoric acid), more preferably dropwise, to the solution. The phosphoric acid may be used in an amount of: about 1.0 mole equivalents to about 3.0 mole equivalents, about 1.2 mole equivalents to about 2.8 mole equivalents, about 1.5 to about 2.5, or about 1.6 to about 2.3 mole equivalents relative to LX9211. The alcohol is preferably a C₁ to C₈ alcohol, more preferably a C₁-C₆ alcohol, most preferably a C₂₋₄ alcohol, and particularly 2-propanol.

In embodiments, Form S1 of LX9211 dihydrogen phosphate as described in any aspect or embodiment of the present disclosure may be prepared by crystallizing LX9211 dihydrogen phosphate in an alcohol or a mixture of an alcohol and water. The mixture of LX9211 dihydrogen phosphate in an alcohol or a mixture of an alcohol and water may be prepared by obtaining a solution of LX9211 dihydrogen phosphate in the alcohol (preferably 2-propanol), and adding water. The solution of LX9211 dihydrogen phosphate in the alcohol may be obtained by dissolving LX9211 in the alcohol and adding phosphoric acid (preferably concentrated phosphoric acid, particularly 85% phosphoric acid), more preferably dropwise, to the solution. The phosphoric acid may be used in an amount of: about 1.8 mole equivalents to about 3.0 mole equivalents, about 2.0 mole equivalents to about 2.5 mole equivalents or about 2.2 mole equivalents relative to LX9211. The alcohol is preferably a C₁ to C₈ alcohol, more preferably a C₁-C₆ alcohol, most preferably a C₂₋₄ alcohol, and particularly 2-propanol. The alcohol (preferably 2-propanol) is preferably used in an amount of: about 1 ml to about 7 ml, about 1 ml to about 6.5 ml, about 2 ml to about 6 ml, about 4 ml to about 5.5 ml, about 4.5 ml to about 5.5 ml, or about 5 ml per gram of LX9211. When a mixture of alcohol and water is used, the water may be in an amount of: about 10 ml to about 40 ml, about 15 ml to about 30 ml, about 15 ml to about 25 ml, or about 20 ml, per gram of LX9211. The alcohol and water may be in a ratio of: about 2:1 to about 1:10, about 1:1 to about 1:8, about 1:1 to about 1:7, about 1:2 to about 1:6, about 1:3 to about 1:5, or about 1:4. The mixture may be stirred, optionally at a temperature of: about 0° C. to about 30° C., about 0° C. to about 20° C., about 0° C. to about 15° C., or about 0° C. to about 5° C. Alternatively, the water may be present in a trace amount (e.g. from the phosphoric acid) for example, about 0.01 ml to about 0.5 ml, about 0.02 ml to about 0.2 ml, about 0.02 ml to about 0.1 ml, about 0.05 ml to about 0.08 ml, or about 0.06 ml, per gram of LX9211. The mixture may be stirred for a sufficient time to prepare Form S1, preferably: about 12 hours to about 5 days, about 16 hours to about 4 days, about 18 hours to about 2 days, or about 20 hours to about 30 hours, or about 24 hours. Form S1 of LX9211 may be isolated by any suitable process, including filtration, decantation or by centrifuge, preferably by filtration. The product may be dried, optionally at: about 20° C. to about 40° C., about 25° C. to about 35° C., or about 30° C. The drying may be carried out for any suitable period of time, particularly about 20 minutes to about 6 hours, about 30 minutes to about 4 hours, about 1 hour to about 3 hours or about 2 hours.

Alternatively, Form S1 of LX9211 dihydrogen phosphate as described in any aspect or embodiment of the present disclosure may be prepared by crystallizing LX9211 dihydrogen phosphate from an alcohol. The mixture of LX9211 dihydrogen phosphate in an alcohol may be prepared by obtaining a solution of LX9211 dihydrogen phosphate in the alcohol (preferably 2-propanol). The solution of LX9211 dihydrogen phosphate in the alcohol may be obtained by dissolving LX9211 in the alcohol and adding phosphoric acid (preferably concentrated phosphoric acid, particularly 85% phosphoric acid), more preferably dropwise, to the solution. The phosphoric acid may be used in an amount of: about 1.2 mole equivalents to about 2.5 mole equivalents, about 1.4 mole equivalents to about 2.2 mole equivalents, about 1.5 mole equivalents to about 1.8 mole equivalents or about 1.7 mole equivalents relative to LX9211. The alcohol is preferably a C₁ to C₈ alcohol, more preferably a C₁-C₆ alcohol, most preferably a C₂₋₄ alcohol, and particularly 2-propanol. The alcohol (preferably 2-propanol) is preferably used in an amount of: about 30 ml to about 70 ml, about 35 ml to about 65 ml, about 40 ml to about 60 ml, about 45 ml to about 55 ml, or about 50 ml per gram of LX9211. Water may be present in a trace amount (e.g. from the phosphoric acid) for example, about 0.01 ml to about 0.5 ml, about 0.02 ml to about 0.2 ml, about 0.02 ml to about 0.05 ml, per gram of LX9211. The suspension may be stirred, preferably at room temperature. Alternatively, the mixture may be prepared by suspending LX9211 dihydrogen phosphate in the alcohol (preferably 2-propanol) and stirring. The stirring may be for a sufficient time to prepare Form S1, preferably: about 1 day to about 7 days, about 2 days to about 6 days, about 3 days to about 5 days, or about 3 days. Form S1 of LX9211 may be isolated by any suitable process, including filtration, decantation or by centrifuge, preferably by filtration. The product may be dried, optionally at: about 20° C. to about 40° C., about 25° C. to about 35° C., or about 30° C. The drying may be carried out for any suitable period of time, particularly about 20 minutes to about 6 hours, about 30 minutes to about 4 hours, about 1 hour to about 3 hours or about 2 hours.

Form S1 of LX9211 dihydrogen phosphate as described in any aspect or embodiment of the present disclosure, may be prepared by slurrying LX9211 dihydrogen phosphate in water. The starting LX9211 dihydrogen phosphate may be any other form of LX9211 dihydrogen phosphate, but is preferably Form S3 as described in any aspect or embodiment of the present disclosure. Preferably, the slurrying comprises stirring LX9211 dihydrogen phosphate in water. The water may be used in an amount of: about 2 ml to about 10 ml, about 4 ml to about 8 ml, about 5 ml to about 6 ml, or about 5 to about 5.5 ml per gram of LX9211 starting material, In any aspect or embodiment of the process, the stirring may be carried out at a temperature of about 10° C. to about 40° C., and preferably at room temperature. The stirring may be carried out for any suitable time to prepare form S1 of LX9211 dihydrogen phosphate. Preferably, the stirring may be for a period of: about 1 hour to about 4 days, about 8 hours to about 3 days, about 12 hours to about 2 days, about 16 hours to about 36 hours, about 20 hours to about 30 hours, or about 24 hours. The reaction mixture of the LX9211 dihydrogen phosphate in water may be formed by combining LX9211 dihydrogen phosphate, preferably Form S3, with the water. The product may be isolated by any suitable procedure, such as decantation, centrifugation or filtration, preferably by filtration. The resulting solid may be dried, optionally under vacuum, for a suitable period of time, preferably about 1 hour to about 10 hours, or about 4 hours to about 8 hours, or about 6 hours. The drying may be carried out at any suitable temperature, preferably: about 25° C. to about 50° C., about 30° C. to about 45° C., about 34° C. to about 45° C., or about 40° C.

The present disclosure further encompasses a product obtainable by any of the above-described processes.

The processes for preparing form S1 of LX9211 dihydrogen phosphate may further comprise combining the Form SI of LX9211 dihydrogen phosphate with at least one pharmaceutically acceptable excipient to form a pharmaceutical composition.

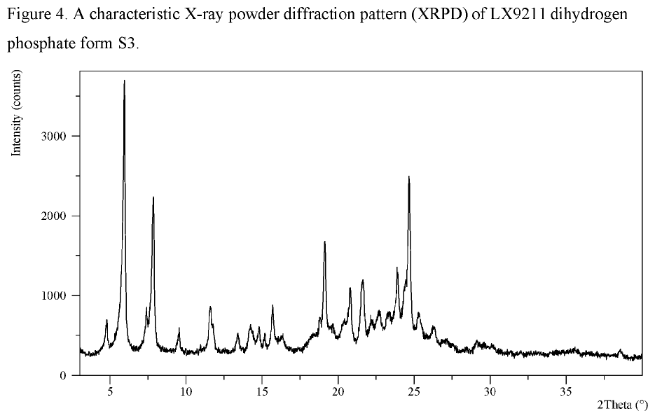

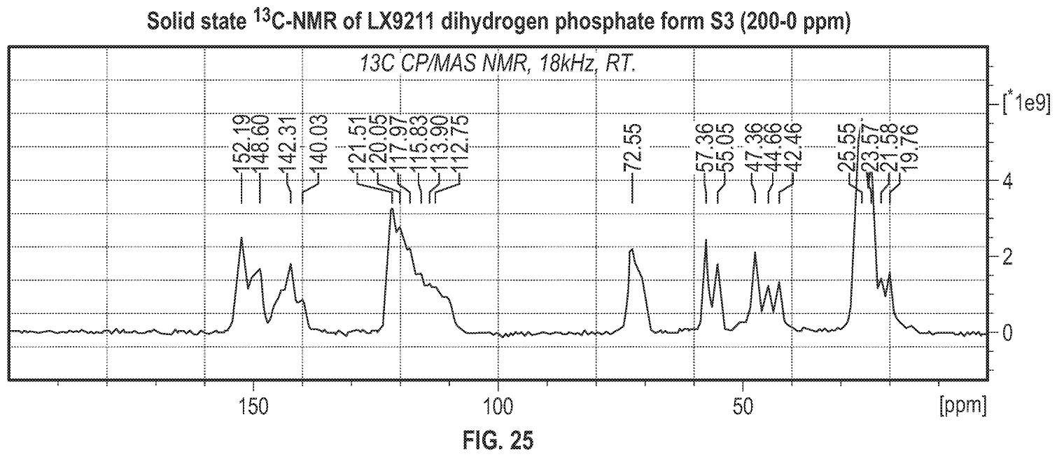

The present disclosure includes a crystalline polymorph LX9211 dihydrogen phosphate designated Form S3. The crystalline Form S3 of LX9211 dihydrogen phosphate may be characterized by data selected from one or more of the following: an X-ray powder diffraction pattern substantially as depicted in FIG. 4; an X-ray powder diffraction pattern having peaks at 4.8, 20.8 and 24.7 degrees 2-theta±0.2 degrees 2-theta; a solid state ¹³C NMR spectrum having characteristic peaks at 142.3, 121.5, 57.4 and 47.4 ppm±0.2 ppm; A solid state ¹³C NMR spectrum having the following chemical shift absolute differences from reference peak at 19.8 ppm±0.2 ppm: 122.5, 101.7, 37.6 and 27.6 ppm±0.1 ppm a solid state ¹³C NMR spectrum substantially as depicted in FIG. 25; and combinations of these data.

Crystalline Form S3 of LX9211 dihydrogen phosphate may be further characterized by an X-ray powder diffraction pattern having peaks at 4.8, 20.8 and 24.7 degrees 2-theta±0.2 degrees 2-theta, and also having any one or both additional peaks selected from 21.6 and 23.9 degrees 2-theta±0.2 degrees 2-theta.

The crystalline Form S3 of LX9211 dihydrogen phosphate may be alternatively characterized by an X-ray powder diffraction pattern having peaks at 4.8, 20.8, 21.6, 23.9 and 24.7±0.2 degrees 2-theta; an XRPD pattern as depicted in FIG. 4 and combinations of these data.

Crystalline Form S3 of LX9211 dihydrogen phosphate may be further characterized by an X-ray powder diffraction pattern having peaks at 4.8, 20.8, 21.6, 23.9 and 24.7±0.2 degrees 2-theta±0.2 degrees 2-theta, and also having any one, two, three, four or five additional peaks selected from 9.5, 11.6, 15.2, 15.7 and 26.3±0.2 degrees 2-theta±0.2 degrees 2-theta.

Crystalline Form S3 of LX9211 dihydrogen phosphate may be alternatively characterized by an X-ray powder diffraction pattern having peaks at 4.8, 9.5, 11.6, 15.2, 15.7, 20.8, 21.6, 23.9, 24.7 and 26.3 degrees 2-theta±0.2 degrees 2-theta.

In any aspect or embodiment of the present disclosure, crystalline Form S3 of LX9211 dihydrogen phosphate is isolated. Particularly, crystalline Form S3 of LX9211 dihydrogen phosphate according to any aspect or embodiment of the disclosure may be isolated.

In any aspect or embodiment crystalline Form S3 of LX9211 dihydrogen phosphate may be chemically pure.

In any aspect or embodiment crystalline Form S3 of LX9211 dihydrogen phosphate may be polymorphically pure.

In any aspect or embodiment of the present disclosure, crystalline form S3 of LX9211 dihydrogen phosphate may be anhydrous.

Crystalline Form S3 of LX9211 dihydrogen phosphate may be characterized by each of the above characteristics alone/or by all possible combinations, e.g., an XRPD pattern having peaks at 4.8, 20.8, 21.6, 23.9 and 24.7 degrees 2-theta±0.2 degrees 2-theta; an XRPD pattern as depicted in FIG. 4, and combinations thereof.

Crystalline Form S3 of LX9211 dihydrogen phosphate according to any aspect or embodiment of the present disclosure may be advantageously stable, for example to conditions of high relative humidity or heating. For example form S3 may show no polymorphic changes when exposed to 80% RH (e.g. at room temperature), e.g. for up to 1 month or under heating to a temperature of about 100° C. e.g. for 30 minutes.

Form S3 of LX9211 dihydrogen phosphate as described in any aspect or embodiment of the present disclosure may be prepared by crystallizing LX9211 dihydrogen phosphate from an alcohol or a mixture of an alcohol and water. The alcohol is preferably a C₁ to C₈ alcohol, more preferably a C₁-C₆ alcohol, most preferably a C₂₋₄ alcohol, and particularly 2-propanol. The mixture of LX9211 dihydrogen phosphate in an alcohol may be prepared by obtaining a solution of LX9211 dihydrogen phosphate in the alcohol (preferably 2-propanol). The solution of LX9211 dihydrogen phosphate in the alcohol may be obtained by dissolving LX9211 in the alcohol and adding phosphoric acid (preferably concentrated phosphoric acid, particularly 85% phosphoric acid), more preferably dropwise, to the solution. The phosphoric acid may be used in an amount of: about 1.2 mole equivalents to about 2.5 mole equivalents, about 1.3 mole equivalents to about 2.2 mole equivalents, about 1.4 mole equivalents to about 1.8 mole equivalents, about 1.5 to about 1.7 mole equivalents, or about 1.6 mole equivalents relative to LX9211. The alcohol (preferably 2-propanol) is preferably used in an amount of: about 3 ml to about 20 ml, about 5 ml to about 18 ml, about 8 ml to about 15 ml, about 8 ml to about 12 ml, or about 10 ml per gram of LX9211. The water may be present in a trace amount (e.g. from the phosphoric acid) for example, about 0.01 ml to about 0.5 ml, about 0.02 ml to about 0.2 ml, about 0.02 ml to about 0.05 ml, or about 0.04 ml, per gram of LX9211. The solution may be stirred, preferably at room temperature. The product may be dried, optionally at: about 20° C. to about 65° C., about 30° C. to about 60° C., about 40° C. to about 55° C. or about 50° C. The drying may be carried out for any suitable period of time, particularly about 20 minutes to about 6 hours, about 30 minutes to about 4 hours, about 45 minutes to about 2 hours, or about 1 hour.

Form S3 of LX9211 dihydrogen phosphate as described in any aspect or embodiment of the present disclosure may be prepared by crystallizing LX9211 dihydrogen phosphate in an alcohol or a mixture of an alcohol and water. The mixture of LX9211 dihydrogen phosphate in an alcohol or a mixture of an alcohol and water may be prepared by obtaining a solution of LX9211 dihydrogen phosphate in the alcohol (preferably 2-propanol), and optionally adding water. The solution of LX9211 dihydrogen phosphate in the alcohol may be obtained by dissolving LX9211 in the alcohol and adding phosphoric acid (preferably concentrated phosphoric acid, particularly 85% phosphoric acid), more preferably dropwise, to the solution. The alcohol is preferably a C₁ to C₈ alcohol, more preferably a C₁-C₆ alcohol, most preferably a C₂₋₄ alcohol, and particularly 2-propanol. The alcohol (preferably 2-propanol) is preferably used in an amount of: about 10 ml to about 60 ml, about 20 ml to about 55 ml, about 30 ml to about 50 ml, about 35 ml to about 45 ml, or about 40 ml per gram of LX9211. The water may be present in a trace amount (e.g. from the phosphoric acid) for example, about 0.01 ml to about 0.5 ml, about 0.02 ml to about 0.2 ml, about 0.02 ml to about 0.1 ml, about 0.05 ml to about 0.08 ml, or about 0.06 ml, per gram of LX9211. The mixture may be stirred, preferably at room temperature. The mixture may be stirred for a sufficient time to prepare Form S3, preferably: about 12 hours to about 7 days, about 24 hours to about 5 days, about 2 days to about 4 days, or about 3 days. Form S3 of LX9211 may be isolated by any suitable process, including filtration, decantation or by centrifuge, preferably by filtration. The product may be dried, optionally at: about 20° C. to about 65° C., about 30° C. to about 60° C., about 40° C. to about 55° C. or about 50° C. The drying may be carried out for any suitable period of time, particularly about 20 minutes to about 6 hours, about 30 minutes to about 4 hours, about 45 minutes to about 2 hours, or about 1 hour.

Form S3 of LX9211 dihydrogen phosphate as described in any aspect or embodiment of the present disclosure, may be prepared by heating LX9211 dihydrogen phosphate. The starting LX9211 dihydrogen phosphate may be any other form of LX9211 dihydrogen phosphate, but is preferably Form S1 as described in any aspect or embodiment of the present disclosure. The heating may be to a temperature of about 80° C. to about 180° C., preferably to about 100° C. to about 170° C., about 120° C. to about 165° C., about 140° C. to about 160° C. or about 150° C. The heating may be performed under vacuum. The heating may be carried out at a rate of about 2° C. to about 20° C., about 5° C. to about 15° C., about 8° C. to about 12° C., or about 10° C., per minute. The heating may be carried stepwise, for example by a two stage process, involving heating to a first temperature of: about 60° C. to about 100° C., or about 70° C. to about 95° C. or about 75° C. to about 90° C., or about 85° C., maintaining the first temperature for a period of: about 5 to about 20 minutes, about 8 to about 15 minutes, or about 10 minutes, optionally cooling to room temperature, and heating through to the final temperature of about 100° C. to about 170° C., about 120° C. to about 165° C., preferably about 140° C. to about 160° C., or about 150° C. The heating may be maintained at the final temperature for a period of: about 5 to about 20 minutes, about 8 to about 15 minutes, or about 10 minutes. The heating may be carried out on a DSC apparatus. The product may be cooled, preferably to room temperature.

The present disclosure further encompasses a product obtainable by any of the above-described processes

The processes for preparing Form S3 of LX9211 dihydrogen phosphate as described in any aspect or embodiment herein may further comprise combining the Form S3 of LX9211 dihydrogen phosphate with at least one pharmaceutically acceptable excipient to form a pharmaceutical composition.

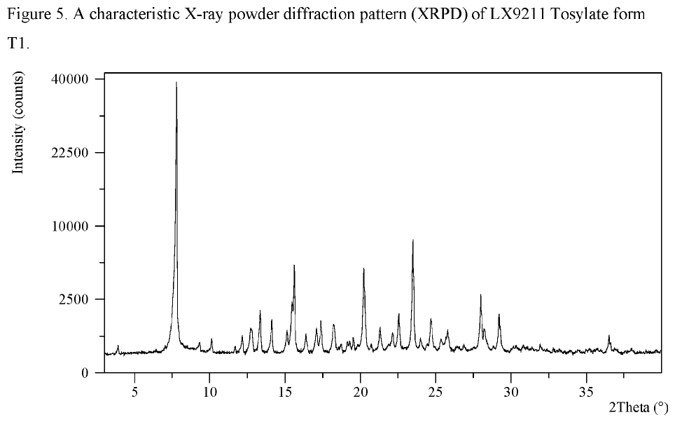

The present disclosure includes a crystalline polymorph LX9211 Tosylate designated Form T1. The crystalline Form T1 of LX9211 Tosylate may be characterized by data selected from one or more of the following: an X-ray powder diffraction pattern substantially as depicted in FIG. 5; an X-ray powder diffraction pattern having peaks at 7.8, 13.4 and 23.5 degrees 2-theta±0.2 degrees 2-theta; and combinations of these data.

Crystalline Form T1 of LX9211 Tosylate may be further characterized by an X-ray powder diffraction pattern having peaks at 7.8, 13.4 and 23.5 degrees 2-theta±0.2 degrees 2-theta, and also having any one or both additional peaks selected from 14.1 and 28.0 degrees 2-theta±0.2 degrees 2-theta.

Crystalline Form T1 of LX9211 Tosylate may be alternatively characterized by an X-ray powder diffraction pattern having peaks at 7.8, 13.4, 14.1, 23.5 and 28.0 degrees 2-theta±0.2 degrees 2-theta.

Crystalline Form T1 of LX9211 Tosylate may be further characterized by an X-ray powder diffraction pattern having peaks at 7.8, 13.4, 14.1, 23.5 and 28.0 degrees 2-theta±0.2 degrees 2-theta, and also having any one, two, three, four or five additional peaks selected from 12.2, 15.6, 17.1, 17.4 and 21.3 degrees 2-theta±0.2 degrees 2-theta.

Crystalline Form T1 of LX9211 Tosylate may be alternatively characterized by an X-ray powder diffraction pattern having peaks at 7.8, 12.2, 13.4, 14.1, 15.6, 17.1, 17.4, 21.3, 23.5 and 28.0 degrees 2-theta±0.2 degrees 2-theta.

In any aspect or embodiment of the present disclosure, crystalline Form T1 of LX9211 Tosylate is isolated. Particularly, crystalline Form T1 of LX9211 Tosylate according to any aspect or embodiment of the disclosure may be isolated.

In any aspect or embodiment crystalline Form TI of LX9211 Tosylate may be polymorphically pure.

Crystalline Form T1 of LX9211 Tosylate may be characterized by each of the above characteristics alone/or by all possible combinations, e.g., an XRPD pattern having peaks at 7.8, 13.4, 14.1, 23.5 and 28.0 degrees 2-theta±0.2 degrees 2-theta; an XRPD pattern as depicted in FIG. 5, and combinations thereof.

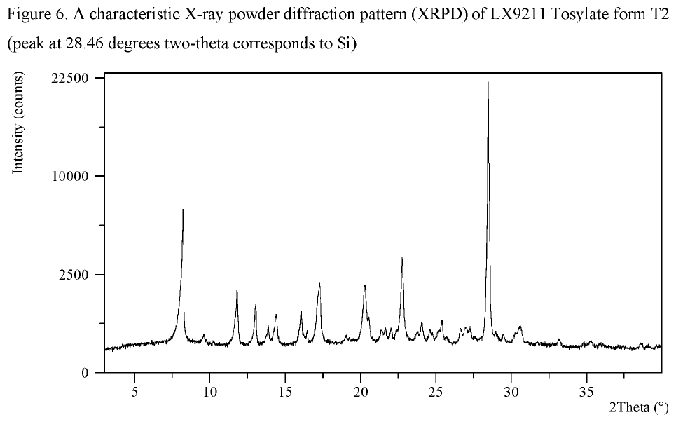

The present disclosure includes a crystalline polymorph LX9211 Tosylate designated Form T2. The crystalline Form T2 of LX9211 Tosylate may be characterized by data selected from one or more of the following: an X-ray powder diffraction pattern substantially as depicted in FIG. 6; an X-ray powder diffraction pattern having peaks at 8.2, 11.8 and 22.8 degrees 2-theta±0.2 degrees 2-theta; and combinations of these data.

Crystalline Form T2 of LX9211 Tosylate may be further characterized by an X-ray powder diffraction pattern having peaks at 8.2, 11.8 and 22.8 degrees 2-theta±0.2 degrees 2-theta, and also having any one or both additional peaks selected from 14.4 and 16.1 degrees 2-theta±0.2 degrees 2-theta.

Crystalline Form T2 of LX9211 Tosylate may be alternatively characterized by an X-ray powder diffraction pattern having peaks at 8.2, 11.8, 14.4, 16.1 and 22.8 degrees 2-theta±0.2 degrees 2-theta.

Crystalline Form T2 of LX9211 Tosylate may be further characterized by an X-ray powder diffraction pattern having peaks at 8.2, 11.8, 14.4, 16.1 and 22.8 degrees 2-theta±0.2 degrees 2-theta, and also having any one, two, three, four or five additional peaks selected from 17.3, 19.0, 20.5, 22.0 and 26.6 degrees 2-theta±0.2 degrees 2-theta.

Crystalline Form T2 of LX9211 Tosylate may be alternatively characterized by an X-ray powder diffraction pattern having peaks at 8.2, 11.8, 14.4, 16.1, 17.3, 19.0, 20.5, 22.0, 22.8, and 26.6 degrees 2-theta±0.2 degrees 2-theta.

In any aspect or embodiment of the present disclosure, crystalline Form T2 of LX9211 Tosylate is isolated. Particularly, crystalline Form T2 of LX9211 Tosylate according to any aspect or embodiment of the disclosure may be isolated.

In any aspect or embodiment crystalline Form T2 of LX9211 Tosylate may be polymorphically pure.

In any aspect or embodiment of the present disclosure, crystalline Form T2 of LX9211 Tosylate may be a hydrate, preferably a monohydrate.

Crystalline Form T2 of LX9211 Tosylate may be characterized by each of the above characteristics alone/or by all possible combinations, e.g., an XRPD pattern having peaks at 8.2, 11.8, 14.4, 16.1 and 22.8 degrees 2-theta±0.2 degrees 2-theta; an XRPD pattern as depicted in FIG. 6, and combinations thereof.

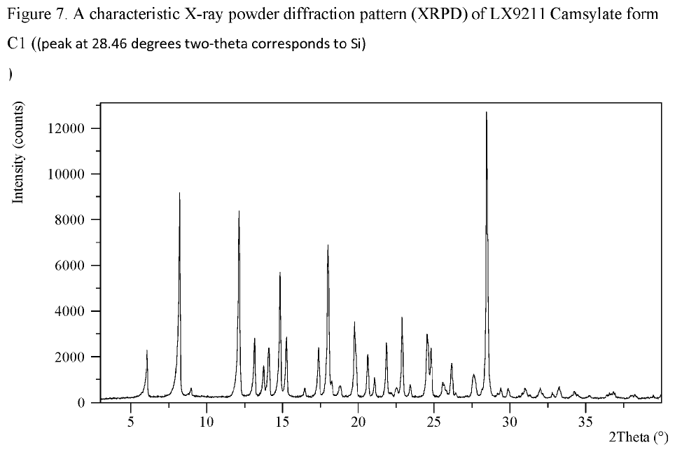

The present disclosure includes a crystalline polymorph LX9211 Camsylate designated Form C1. The crystalline Form C1 of LX9211 Camsylate may be characterized by data selected from one or more of the following: an X-ray powder diffraction pattern substantially as depicted in FIG. 7; an X-ray powder diffraction pattern having peaks at 12.1, 14.8 and 18.0 degrees 2-theta±0.2 degrees 2-theta; and combinations of these data.

Crystalline Form C1 of LX9211 camsylate may be further characterized by an X-ray powder diffraction pattern having peaks at 12.1, 14.8 and 18.0 degrees 2-theta±0.2 degrees 2-theta, and also having any one or both additional peaks selected from 8.2 and 19.7 degrees 2-theta±0.2 degrees 2-theta.

Crystalline Form C1 of LX9211 camsylate may be alternatively characterized by an X-ray powder diffraction pattern having peaks at 8.2, 12.1, 14.8, 18.0 and 19.7 degrees 2-theta±0.2 degrees 2-theta.

Crystalline Form C1 of LX9211 camsylate may be further characterized by an X-ray powder diffraction pattern having peaks at 8.2, 12.1, 14.8, 18.0 and 19.7 degrees 2-theta±0.2 degrees 2-theta, and also having any one, two, three, four or five additional peaks selected from 6.1, 14.1, 20.6, 21.9 and 26.1 degrees 2-theta±0.2 degrees 2-theta.

Crystalline Form C1 of LX9211 camsylate may be alternatively characterized by an X-ray powder diffraction pattern having peaks at 6.1, 8.2, 12.1, 14.1, 14.8, 18.0, 19.7, 20.6, 21.9 and 26.1 degrees 2-theta±0.2 degrees 2-theta.

In any aspect or embodiment of the present disclosure, crystalline Form C1 of LX9211 camsylate is isolated. Particularly, crystalline Form C1 of LX9211 camsylate according to any aspect or embodiment of the disclosure may be isolated.

In any aspect or embodiment crystalline Form C1 of LX9211 camsylate may be polymorphically pure.

In any aspect or embodiment of the present disclosure, crystalline Form C1 of LX9211 camsylate may be anhydrous.

Crystalline Form C1 of LX9211 camsylate may be characterized by each of the above characteristics alone/or by all possible combinations, e.g., an XRPD pattern having peaks at 8.2, 12.1, 14.8, 18.0 and 19.7 degrees 2-theta±0.2 degrees 2-theta; an XRPD pattern as depicted in FIG. 7, and combinations thereof.

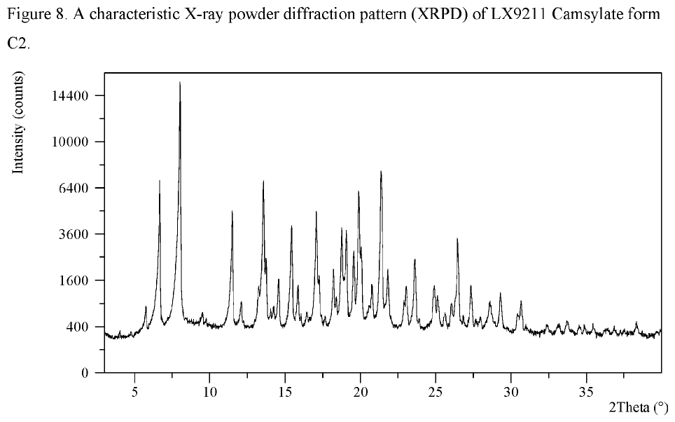

The present disclosure includes a crystalline polymorph LX9211 Camsylate designated Form C2. The crystalline Form C2 of LX9211 Camsylate may be characterized by data selected from one or more of the following: an X-ray powder diffraction pattern substantially as depicted in FIG. 8; an X-ray powder diffraction pattern having peaks at 6.7, 21.4 and 26.5 degrees 2-theta±0.2 degrees 2-theta; and combinations of these data.

Crystalline Form C2 of LX9211 camsylate may be further characterized by an X-ray powder diffraction pattern having peaks at 6.7, 21.4 and 26.5 degrees 2-theta±0.2 degrees 2-theta, and also having any one or both additional peaks selected from 13.6 and 17.1 degrees 2-theta±0.2 degrees 2-theta.

Crystalline Form C2 of LX9211 camsylate may be alternatively characterized by an X-ray powder diffraction pattern having peaks at 6.7, 13.6, 17.1, 21.4 and 26.5 degrees 2-theta±0.2 degrees 2-theta.

Crystalline Form C2 of LX9211 camsylate may be further characterized by an X-ray powder diffraction pattern having peaks at 6.7, 13.6, 17.1, 21.4 and 26.5 degrees 2-theta±0.2 degrees 2-theta, and also having any one, two, three, four, and/or five additional peaks selected from 11.5, 14.6, 15.4, 15.9, and 23.6 degrees 2-theta±0.2 degrees 2-theta.

Crystalline Form C1 of LX9211 camsylate may be alternatively characterized by an X-ray powder diffraction pattern having peaks at 6.7, 11.5, 13.6, 14.6, 15.4, 15.9, 17.1, 21.4, 23.6 and 26.5 degrees 2-theta±0.2 degrees 2-theta.

In any aspect or embodiment of the present disclosure, crystalline Form C2 of LX9211 camsylate is isolated. Particularly, crystalline Form C2 of LX9211 camsylate according to any aspect or embodiment of the disclosure may be isolated.

In any aspect or embodiment crystalline Form C2 of LX9211 camsylate may be polymorphically pure.

In any aspect or embodiment of the present disclosure, crystalline Form C2 of LX9211 camsylate may be anhydrous.

Crystalline Form C2 of LX9211 camsylate may be characterized by each of the above characteristics alone/or by all possible combinations, e.g., an XRPD pattern having peaks at 6.7, 13.6, 17.1, 21.4 and 26.5 degrees 2-theta±0.2 degrees 2-theta; an XRPD pattern as depicted in FIG. 8, and combinations thereof.

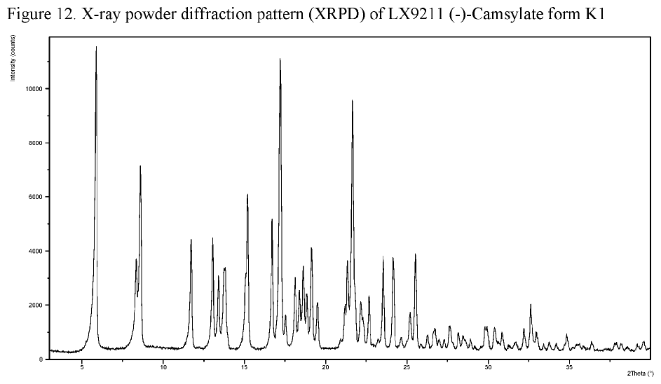

The present disclosure includes a crystalline polymorph LX9211 (−)-Camsylate designated Form K1. The crystalline Form K1 of LX9211 (−)-Camsylate may be characterized by data selected from one or more of the following: an X-ray powder diffraction pattern substantially as depicted in FIG. 12; an X-ray powder diffraction pattern having peaks at 5.9, 11.7 and 21.7 degrees 2-theta±0.2 degrees 2-theta; and combinations of these data.

Crystalline Form K1 of LX9211 (−)-camsylate may be further characterized by an X-ray powder diffraction pattern having peaks at 5.9, 11.7 and 21.7 degrees 2-theta±0.2 degrees 2-theta, and also having any one or both additional peaks selected from 8.6 and 23.5 degrees 2-theta±0.2 degrees 2-theta.

Crystalline Form K1 of LX9211 (−)-camsylate may be alternatively characterized by an X-ray powder diffraction pattern having peaks at 5.9, 8.6, 11.7, 21.7 and 23.5 degrees 2-theta±0.2 degrees 2-theta.

Crystalline Form C1 of LX9211 (−)-camsylate may be further characterized by an X-ray powder diffraction pattern having peaks at 5.9, 8.6, 11.7, 21.7 and 23.5 degrees 2-theta±0.2 degrees 2-theta, and also having any one, two, three, four or five additional peaks selected from 8.3, 13.4, 18.1, 25.2 and 25.5 degrees 2-theta±0.2 degrees 2-theta.

Crystalline Form K1 of LX9211 (−)-camsylate may be alternatively characterized by an X-ray powder diffraction pattern having peaks at 5.9, 8.3, 8.6, 11.7, 13.4, 18.1, 21.7, 23.5, 25.2 and 25.5 degrees 2-theta±0.2 degrees 2-theta.

In any aspect or embodiment of the present disclosure, crystalline Form K1 of LX9211 (−)-camsylate is isolated. Particularly, crystalline Form K1 of LX9211 (−)-camsylate according to any aspect or embodiment of the disclosure may be isolated.

In any aspect or embodiment crystalline Form K1 of LX9211 (−)-camsylate may be chemically pure.

In any aspect or embodiment crystalline Form K1 of LX9211 (−)-camsylate may be polymorphically pure.

In any aspect or embodiment of the present disclosure, crystalline Form K1 of LX9211 (−)-camsylate may be anhydrous.

Crystalline Form K1 of LX9211 (−)-camsylate may be characterized by each of the above characteristics alone/or by all possible combinations, e.g., an XRPD pattern having peaks at 5.9, 8.6, 11.7, 21.7 and 23.5 degrees 2-theta±0.2 degrees 2-theta; an XRPD pattern as depicted in FIG. 12, and combinations thereof.

Crystalline Form K1 of LX9211 (−)-camsylate according to any aspect or embodiment of the present disclosure may be advantageously stable, for example to conditions of high relative humidity or heating. For example form K1 may show no polymorphic changes when exposed to 100% RH (e.g. at room temperature), e.g. for up to 1 month or under heating to a temperature of about 100° C. e.g. for 30 minutes.

The present disclosure includes a crystalline polymorph of LX9211: phosphoric acid designated Form S5. As used herein, Form S5 of LX9211: phosphoric acid is a crystalline solid that comprises LX9211 and phosphoric acid. In embodiments the molar ratio between the active pharmaceutical ingredient (LX9211) and phosphoric acid is about 2:1. In embodiments, form S5 LX9211: phosphoric acid may be a salt. In embodiments form S5 may be a hemihydrogen phosphate salt of LX9211 (i.e. [LX9211]2HPO4). Alternatively form S5 may be a co-crystal. In embodiments form S5 may be a co-crystal of LX9211 dihydrogen phosphate such as a co-crystal of LX9211 dihydrogen phosphate with LX9211. Preferably in any aspect or embodiment of the present disclosure form S5 is a hemihydrogen phosphate salt of LX9211 (i.e. [LX9211]2HPO4).

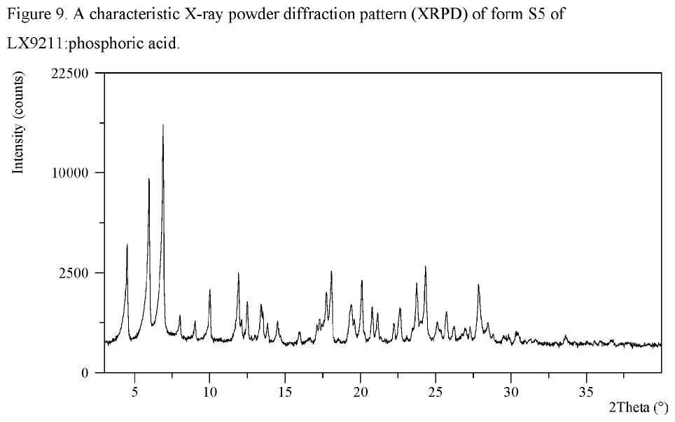

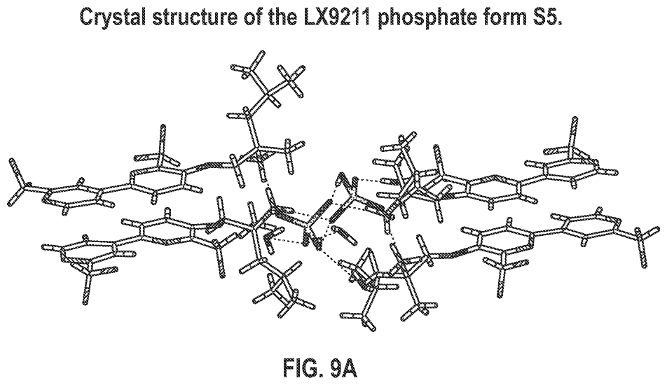

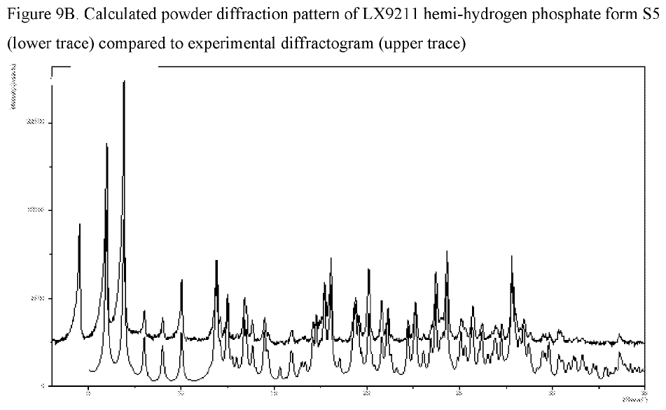

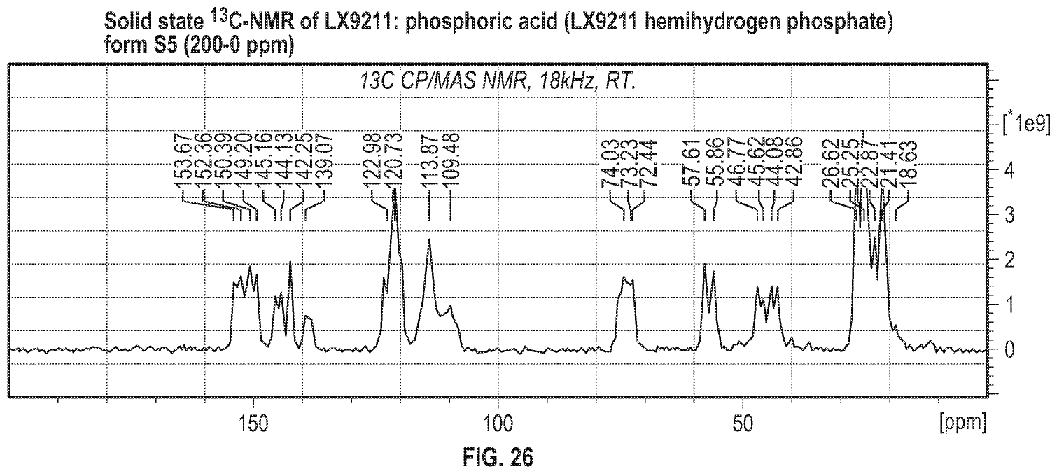

The crystalline Form S5 of LX9211: phosphoric acid may be characterized by data selected from one or more of the following: an X-ray powder diffraction pattern substantially as depicted in FIG. 9; an X-ray powder diffraction pattern having peaks at 4.5, 11.9 and 18.1 degrees 2-theta±0.2 degrees 2-theta; a solid state ¹³C NMR spectrum having characteristic peaks at 150.4, 123.0, 113.9 and 26.6 ppm±0.2 ppm; A solid state ¹³C NMR spectrum having the following chemical shift absolute differences from reference peak at 21.4 ppm±0.2 ppm: 129.0, 101.6, 92.5 and 5.2 ppm±0.1 ppm a solid state ¹³C NMR spectrum substantially as depicted in FIG. 26; and combinations of these data.

Crystalline Form S5 of LX9211: phosphoric acid may be further characterized by an X-ray powder diffraction pattern having peaks at 4.5, 11.9 and 18.1 degrees 2-theta±0.2 degrees 2-theta, and also having any one or both additional peaks selected from 6.9 and 10.0 degrees 2-theta±0.2 degrees 2-theta.

Crystalline Form S5 of LX9211: phosphoric acid may be alternatively characterized by an X-ray powder diffraction pattern having peaks at 4.5, 6.9, 10.0, 11.9 and 18.1 degrees 2-theta±0.2 degrees 2-theta.

Crystalline Form S5 of LX9211: phosphoric acid may be further characterized by an X-ray powder diffraction pattern having peaks at 4.5, 6.9, 10.0, 11.9 and 18.1 degrees 2-theta±0.2 degrees 2-theta, and also having any one, two, three, four or five additional peaks selected from 12.5, 17.7, 20.1, 25.7 and 27.8 degrees 2-theta±0.2 degrees 2-theta.

Crystalline Form S5 of LX9211: phosphoric acid may be alternatively characterized by an X-ray powder diffraction pattern having peaks at 4.5, 6.9, 10.0, 11.9, 12.5, 17.7, 18.1, 20.1, 25.7 and 27.8 degrees 2-theta±0.2 degrees 2-theta.

In any aspect or embodiment of the present disclosure, crystalline Form S5 of LX9211: phosphoric acid is isolated. Particularly, crystalline Form S5 of LX9211: phosphoric acid according to any aspect or embodiment of the disclosure may be isolated.

In any aspect or embodiment crystalline Form S5 of LX9211: phosphoric acid may be polymorphically pure.

In any embodiment of the present disclosure, crystalline Form S5 of LX9211: phosphoric acid may be a hydrate, preferably a monohydrate. In any embodiment, crystalline Form S5 of LX9211 hemi-hydrogen phosphate may contain from about 2% to about 6% of water, preferably about 3% to about 5%, particularly about 4% of water, as measured by TGA.

Alternatively, the present disclosure provides form S5 of LX9211: phosphoric acid can be characterized by the following unit cell data:

| cell length a | 7.44 | A |
|---|---|---|
| cell length b | 14.94 | A |
| cell length c | 19.81 | A |
| cell angle alpha | 81.4° | |
| cell angle beta | 85.9° | |
| cell angle gamma | 83.6° | |
| cell volume | 2158.05 | A³ |

symmetry cell setting triclinic
symmetry space group name P1

Cell data is preferably measured at 170K. Alternatively, form S5 of LX9211: phosphoric acid as defined in any aspect or embodiment herein may be additionally characterized by the above unit cell data.

Crystalline Form S5 of LX9211: phosphoric acid may be characterized by each of the above characteristics alone/or by all possible combinations, e.g., an XRPD pattern having peaks at 4.5, 6.9, 10.0, 11.9 and 18.1 degrees 2-theta±0.2 degrees 2-theta; an XRPD pattern as depicted in FIG. 9, and combinations thereof.

Crystalline Form S5 of LX9211: phosphoric acid according to any aspect or embodiment of the present disclosure may be advantageously stable, for example to conditions of high relative humidity. For example, form S5 may show no polymorphic changes when exposed to 100% RH (e.g. at room temperature), e.g. for up to 1 month.

Form S5 of LX9211 hemihydrogen phosphate as described in any aspect or embodiment of the present disclosure may be prepared by crystallizing LX9211 dihydrogen phosphate in a ketone or mixture of a ketone and water. The ketone is preferably a C₃ to C₈ ketone, or a C₃ to C₅ ketone, and more preferably acetone. The process may comprise obtaining a solution of LX9211 hemihydrogen phosphate in the ketone, or mixture of the ketone and water (preferably wherein the ketone solvent is acetone) and crystallising, preferably at room temperature. The solution of LX9211 hemihydrogen phosphate in the ketone (preferably acetone) or the mixture of the ketone solvent and water may be obtained by combining a mixture of LX9211 in the ketone or in a mixture of ketone and water with phosphoric acid, (preferably concentrated phosphoric acid, particularly 85% phosphoric acid), more preferably dropwise to the solution. The phosphoric acid may be used in an amount of: about 0.3 mole equivalents to about 1.2 mole equivalents, about 0.4 mole equivalents to about 1.0 mole equivalents, relative to LX9211.

Form S5 of LX9211 hemihydrogen phosphate as described in any aspect or embodiment of the present disclosure may be prepared by crystallizing LX9211 dihydrogen phosphate in a mixture of a ketone and water. The solution of LX9211 hemihydrogen phosphate in the mixture of the ketone and water may be obtained by dissolving LX9211 in the ketone and water mixture, and adding phosphoric acid (preferably concentrated phosphoric acid, particularly 85% phosphoric acid), more preferably dropwise, to the solution. The ratio (v/v) of ketone to water may be: about 3:1 to about 1:3, about 2:1 to about 1:3, about 1:1 to about 1:3, about 1:1.5 to about 1:2.5, about 1:1.8 to about 1:2.2, or about 1:2. The mixture of ketone and water may be used in an amount of: about 3 ml to about 20 ml, about 4 ml to about 15 ml, about 5 ml to about 10 ml, about 5 ml to about 8 ml, or about 6 ml to about 7 ml, per gram of LX9211. The phosphoric acid may be used in an amount of: about 0.35 to about 0.6 mole equivalents, about 0.36 mole equivalents to about 0.5 mole equivalents, about 0.37 mole equivalents to about 0.45 mole equivalents, about 0.38 mole equivalents to about 0.42 mole equivalents, or about 0.4 mole equivalents. The solution may be stirred, preferably at room temperature. The product may be isolated by any suitable procedure, such as decantation, centrifugation or filtration, preferably by filtration. The resulting solid may be dried, optionally under vacuum for a suitable period of time. The process may further comprise combining the Form S5 of LX9211 hemihydrogen phosphate with at least one pharmaceutically acceptable excipient to form a pharmaceutical composition.

Form S5 of LX9211 hemihydrogen phosphate as described in any aspect or embodiment of the present disclosure may be prepared by crystallizing LX9211 dihydrogen phosphate in a ketone. The ketone is preferably a C₃ to C₅ ketone, or a C₃ to C₆ ketone, more preferably acetone. The solution of LX9211 hemihydrogen phosphate in the mixture of the ketone and water may be obtained by dissolving LX9211 in the ketone, and adding phosphoric acid (preferably concentrated phosphoric acid, particularly 85% phosphoric acid), more preferably dropwise, to the solution. The ketone may be used in an amount of: about 3 ml to about 20 ml, about 4 ml to about 15 ml, about 5 ml to about 10 ml, about 5 ml to about 8 ml, or about 6 ml to about 7 ml, per gram of LX9211. The phosphoric acid may be used in an amount of: about 0.4 to about 1.2 mole equivalents, about 0.5 mole equivalents to about 1.1 mole equivalents, about 0.7 mole equivalents to about 1.0 mole equivalents, or about 0.9 mole equivalents. The solution may be stirred, preferably at room temperature. The product may be isolated by any suitable procedure, such as decantation, centrifugation or filtration, preferably by filtration. The resulting solid may be dried, optionally under vacuum for a suitable period of time. The solution of LX9211 hemihydrogen phosphate in the ketone (preferably acetone) or the mixture of the ketone solvent and water may be obtained by combining a mixture of LX9211 in the ketone or in a mixture of ketone and water with phosphoric acid. The process may further comprise combining the Form S5 of LX9211 hemihydrogen phosphate with at least one pharmaceutically acceptable excipient to form a pharmaceutical composition.

The present disclosure further encompasses a product obtainable by any of the above-described processes.

The processes for preparing Form S5 of LX9211 dihydrogen phosphate as described in any aspect or embodiment herein may further comprise combining the Form S5 of LX9211 dihydrogen phosphate with at least one pharmaceutically acceptable excipient to form a pharmaceutical composition.

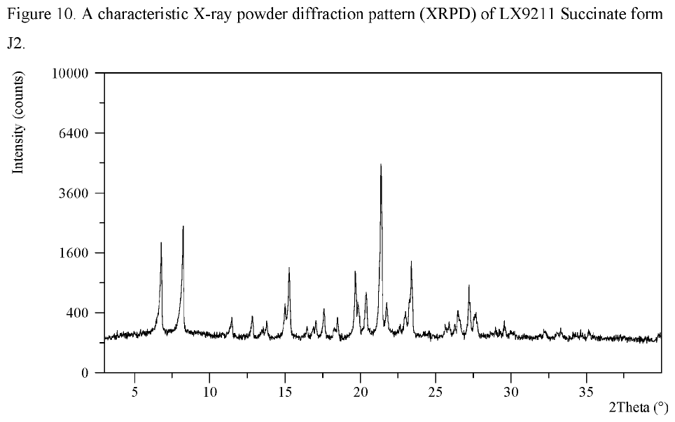

The present disclosure includes a crystalline polymorph LX9211 Succinate designated Form J2. The crystalline Form J2 of LX9211 Succinate may be characterized by data selected from one or more of the following: an X-ray powder diffraction pattern substantially as depicted in FIG. 10; an X-ray powder diffraction pattern having peaks at 6.8, 8.2 and 21.4 degrees 2-theta±0.2 degrees 2-theta; and combinations of these data.

Crystalline Form J2 of LX9211 Succinate may be further characterized by an X-ray powder diffraction pattern having peaks at 6.8, 8.2 and 21.4 degrees 2-theta±0.2 degrees 2-theta, and also having any one or both additional peaks selected from 15.3 and 23.4 degrees 2-theta±0.2 degrees 2-theta.

Crystalline Form J2 of LX9211 Succinate may be alternatively characterized by an X-ray powder diffraction pattern having peaks at 6.8, 8.2, 15.3, 21.4 and 23.4 degrees 2-theta±0.2 degrees 2-theta.

Crystalline Form J2 of LX9211 Succinate may be further characterized by an X-ray powder diffraction pattern having peaks at 6.8, 8.2, 15.3, 21.4, and 23.4 degrees 2-theta±0.2 degrees 2-theta, and also having any one, two, three, four, and/or five additional peaks selected from 12.8, 19.7, 19.8, 20.4 and 26.5 degrees 2-theta±0.2 degrees 2-theta.

Crystalline Form J2 of LX9211 Succinate may be alternatively characterized by an X-ray powder diffraction pattern having peaks at 6.8, 8.2, 12.8, 15.3, 19.7, 19.8, 20.4, 21.4, 23.4 and 26.5 degrees 2-theta±0.2 degrees 2-theta.

In any aspect or embodiment of the present disclosure, crystalline Form J2 of LX9211 Succinate is isolated. Particularly, crystalline Form J2 of LX9211 Succinate according to any aspect or embodiment of the disclosure may be isolated.

In any aspect or embodiment crystalline Form J2 of LX9211 Succinate may be polymorphically pure.

In any aspect or embodiment of the present disclosure, crystalline Form J2 of LX9211 Succinate may be anhydrous.

Crystalline Form J2 of LX9211 Succinate may be characterized by each of the above characteristics alone/or by all possible combinations, e.g., an XRPD pattern having peaks at 6.8, 8.2, 15.3, 21.4, and 23.4 degrees 2-theta±0.2 degrees 2-theta; an XRPD pattern as depicted in FIG. 10, and combinations thereof.

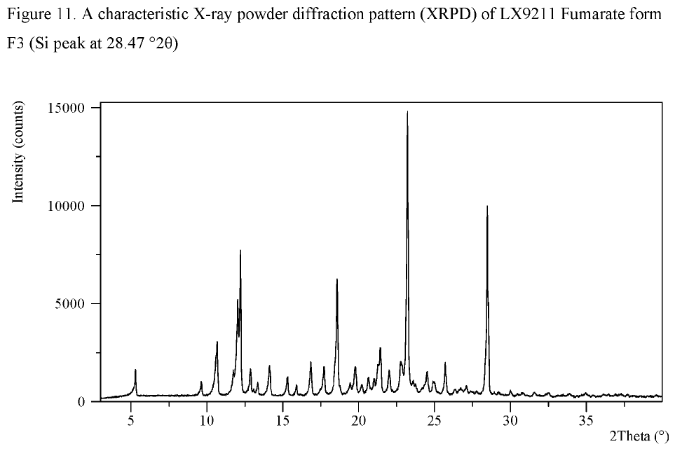

The present disclosure includes a crystalline polymorph LX9211 Fumarate designated Form F3. The crystalline Form F3 of LX9211 Fumarate may be characterized by data selected from one or more of the following: an X-ray powder diffraction pattern substantially as depicted in FIG. 11; an X-ray powder diffraction pattern having peaks at 5.3, 10.7 and 23.2 degrees 2-theta±0.2 degrees 2-theta; and combinations of these data.

Crystalline Form F3 of LX9211 Fumarate may be further characterized by an X-ray powder diffraction pattern having peaks at 5.3, 10.7 and 23.2 degrees 2-theta±0.2 degrees 2-theta, and also having any one or both additional peaks selected from 12.2 and 16.8 degrees 2-theta±0.2 degrees 2-theta.

Crystalline Form F3 of LX9211 Fumarate may be alternatively characterized by an X-ray powder diffraction pattern having peaks at 5.3, 10.7, 12.2, 16.8 and 23.2 degrees 2-theta±0.2 degrees 2-theta.

Crystalline Form F3 of LX9211 Fumarate may be further characterized by an X-ray powder diffraction pattern having peaks at 5.3, 10.7, 12.2, 16.8 and 23.2 degrees 2-theta±0.2 degrees 2-theta, and also having any one, two, three, four, and/or five additional peaks selected from 9.6, 12.0, 19.8, 21.4 and 22.0 degrees 2-theta±0.2 degrees 2-theta.

Crystalline Form F3 of LX9211 Fumarate may be alternatively characterized by an X-ray powder diffraction pattern having peaks at 5.3, 9.6, 10.7, 12.0, 12.2, 16.8, 19.8, 21.4, 22.0 and 23.2 degrees 2-theta±0.2 degrees 2-theta.

In any aspect or embodiment of the present disclosure, crystalline Form F3 of LX9211 Fumarate is isolated. Particularly, crystalline Form F3 of LX9211 Fumarate according to any aspect or embodiment of the disclosure may be isolated.

In any aspect or embodiment crystalline Form F3 of LX9211 Fumarate may be polymorphically pure.

In any aspect or embodiment of the present disclosure, crystalline Form F3 of LX9211 Fumarate may be anhydrous.

Crystalline Form F3 of LX9211 Fumarate may be characterized by each of the above characteristics alone/or by all possible combinations, e.g., an XRPD pattern having peaks at 5.3, 10.7, 12.2, 16.8 and 23.2 degrees 2-theta±0.2 degrees 2-theta; an XRPD pattern as depicted in FIG. 11, and combinations thereof.

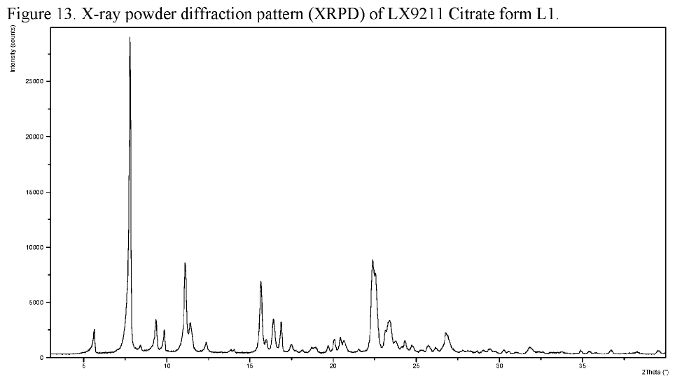

The present disclosure includes a crystalline polymorph LX9211 Citrate designated Form L1. The crystalline Form L1 of LX9211 Citrate may be characterized by data selected from one or more of the following: an X-ray powder diffraction pattern substantially as depicted in FIG. 13; an X-ray powder diffraction pattern having peaks at 5.6, 7.8 and 9.9 degrees 2-theta±0.2 degrees 2-theta; and combinations of these data.

Crystalline Form L1 of LX9211 Citrate may be further characterized by an X-ray powder diffraction pattern having peaks at 5.6, 7.8 and 9.9 degrees 2-theta±0.2 degrees 2-theta, and also having any one or both additional peaks selected from 11.1 and 15.7 degrees 2-theta±0.2 degrees 2-theta.

Crystalline Form L1 of LX9211 Citrate may be alternatively characterized by an X-ray powder diffraction pattern having peaks at 5.6, 7.8, 9.9, 11.1 and 15.7 degrees 2-theta±0.2 degrees 2-theta.

Crystalline Form L1 of LX9211 Citrate may be further characterized by an X-ray powder diffraction pattern having peaks at 5.6, 7.8, 9.9, 11.1 and 15.7 degrees 2-theta±0.2 degrees 2-theta, and also having any one, two, three, four, and/or five additional peaks selected from 16.0, 19.7, 20.6, 22.4 and 26.8 degrees 2-theta±0.2 degrees 2-theta.

Crystalline Form L1 of LX9211 Citrate may be alternatively characterized by an X-ray powder diffraction pattern having peaks at 5.6, 7.8, 9.9, 11.1, 15.7, 16.0, 19.7, 20.6, 22.4 and 26.8 degrees 2-theta±0.2 degrees 2-theta.

In any aspect or embodiment of the present disclosure, crystalline Form L1 of LX9211 Citrate is isolated. Particularly, crystalline Form L1 of LX9211 Citrate according to any aspect or embodiment of the disclosure may be isolated.

In any aspect or embodiment of the present disclosure crystalline Form L1 of LX9211 Citrate may be polymorphically pure.

In any aspect or embodiment of the present disclosure, crystalline Form L1 of LX9211 Citrate may be an acetonitrile solvate.

Crystalline Form L1 of LX9211 Citrate may be characterized by each of the above characteristics alone/or by all possible combinations, e.g., an XRPD pattern having peaks at 5.6, 7.8, 9.9, 11.1 and 15.7 degrees 2-theta±0.2 degrees 2-theta; an XRPD pattern as depicted in FIG. 13, and combinations thereof.

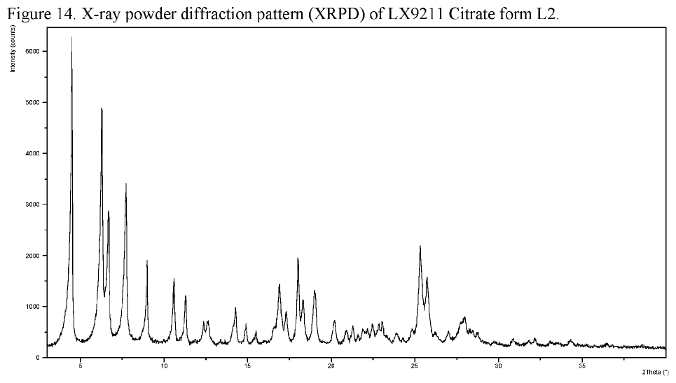

The present disclosure includes a crystalline polymorph LX9211 Citrate designated Form L2. The crystalline Form L2 of LX9211 Citrate may be characterized by data selected from one or more of the following: an X-ray powder diffraction pattern substantially as depicted in FIG. 14; an X-ray powder diffraction pattern having peaks at 4.5, 9.0 and 10.6 degrees 2-theta±0.2 degrees 2-theta; and combinations of these data.

Crystalline Form L2 of LX9211 Citrate may be further characterized by an X-ray powder diffraction pattern having peaks at 4.5, 9.0 and 10.6 degrees 2-theta±0.2 degrees 2-theta, and also having any one or both additional peaks selected from 6.3 and 6.7 degrees 2-theta±0.2 degrees 2-theta.

Crystalline Form L2 of LX9211 Citrate may be alternatively characterized by an X-ray powder diffraction pattern having peaks at 4.5, 6.3, 6.7, 9.0 and 10.6 degrees 2-theta±0.2 degrees 2-theta.

Crystalline Form L2 of LX9211 Citrate may be further characterized by an X-ray powder diffraction pattern having peaks at 4.5, 6.3, 6.7, 9.0 and 10.6 degrees 2-theta±0.2 degrees 2-theta, and also having any one, two, three, four, and/or five additional peaks selected from 12.4, 14.3, 16.9, 21.3 and 25.3 degrees 2-theta±0.2 degrees 2-theta.

Crystalline Form L2 of LX9211 Citrate may be alternatively characterized by an X-ray powder diffraction pattern having peaks at 4.5, 6.3, 6.7, 9.0, 10.6, 12.4, 14.3, 16.9, 21.3 and 25.3 degrees 2-theta±0.2 degrees 2-theta.

In any aspect or embodiment of the present disclosure, crystalline Form L2 of LX9211 Citrate is isolated. Particularly, crystalline Form L2 of LX9211 Citrate according to any aspect or embodiment of the disclosure may be isolated.

In any aspect or embodiment of the present disclosure crystalline Form L2 of LX9211 Citrate may be polymorphically pure.

In any aspect or embodiment of the present disclosure, crystalline Form L2 of LX9211 Citrate may be anhydrous.

Crystalline Form L2 of LX9211 Citrate may be characterized by each of the above characteristics alone/or by all possible combinations, e.g., an XRPD pattern having peaks at 4.5, 6.3, 6.7, 9.0 and 10.6 degrees 2-theta±0.2 degrees 2-theta; an XRPD pattern as depicted in FIG. 14, and combinations thereof.

The present disclosure includes crystalline polymorphs of LX9211 Oxalate designated Form O5 and Form O2. The crystalline Form O5 and Form O2 of LX9211 Oxalate may be characterized by an X-ray powder diffraction pattern having peaks at 4.4, 6.4 and 25.3 degrees 2-theta±0.2 degrees 2-theta.

Crystalline Form O5 and Form O2 of LX9211 Oxalate may be further characterized by an X-ray powder diffraction pattern having peaks at 4.4, 6.4 and 25.3 degrees 2-theta±0.2 degrees 2-theta, and also having any one or both additional peaks selected from 7.2 and 8.8 degrees 2-theta±0.2 degrees 2-theta.

Crystalline Form O5 and Form O2 of LX9211 Oxalate may be alternatively characterized by an X-ray powder diffraction pattern having peaks at 4.4, 6.4, 7.2, 8.8 and 25.3 degrees 2-theta±0.2 degrees 2-theta.

Crystalline Form O5 and Form O2 of LX9211 Oxalate may be further characterized by an X-ray powder diffraction pattern having peaks at 4.4, 6.4, 7.2, 8.8 and 25.3 degrees 2-theta±0.2 degrees 2-theta, and also having any one, two, three, four, and/or five additional peaks selected from 8.3, 11.3, 17.4, 19.6 and 20.5 degrees 2-theta±0.2 degrees 2-theta. Crystalline Forms O5 and Form O2 LX9211 Oxalate may be alternatively characterized by an X-ray powder diffraction pattern having peaks at 4.4, 6.4, 7.2, 8.3, 8.8, 11.3, 17.4, 19.6, 20.5 and 25.3 degrees 2-theta±0.2 degrees 2-theta.

Alternatively, the present disclosure provides crystalline forms of LX9211 Oxalate, preferably Form O5 or Form O2, which may be characterized by X-ray powder diffraction pattern having peaks at 6.3, 7.2, 8.2, 11.3 and 25.3 degrees 2-theta±0.2 degrees 2-theta.

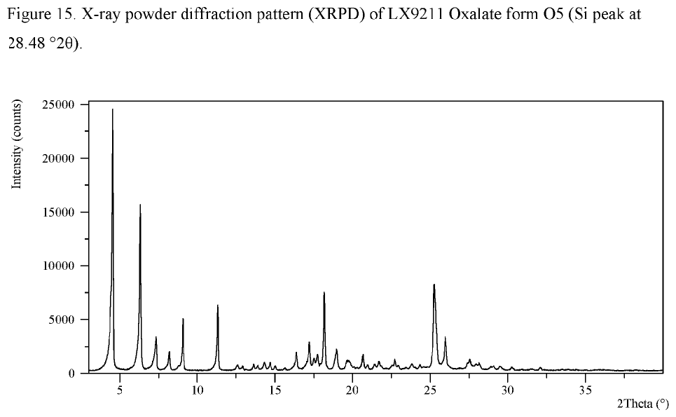

Thus, in an alternative embodiment, crystalline form O5 of LX9211 Oxalate may be characterized by data selected from one or more of the following: an X-ray powder diffraction pattern substantially as depicted in FIG. 15; an X-ray powder diffraction pattern having peaks at 6.3, 7.2, 8.2, 9.1, 11.3, 17.2, 18.2 and 25.3 degrees 2-theta±0.2 degrees 2-theta. Crystalline form O5 of LX9211 Oxalate may be further characterized an X-ray powder diffraction pattern having peaks at 6.3, 7.2, 8.2, 9.1, 11.3, 17.2, 18.2 and 25.3 degrees 2-theta±0.2 degrees 2-theta and also having any one, two or three additional peaks selected from 4.6, 14.7 and 20.7 degrees 2-theta±0.2 degrees 2-theta. Crystalline form O5 of LX9211 Oxalate may be alternatively characterized by an X-ray powder diffraction pattern having peaks at 4.6, 6.3, 7.2, 8.2, 9.1, 11.3, 14.7, 17.2, 18.2, 20.7 and 25.3 degrees 2-theta±0.2 degrees 2-theta.

Crystalline form O5 of LX9211 Oxalate according to any aspect or embodiment as described herein may be isolated. Particularly, crystalline form O5 of LX9211 Oxalate according to any aspect or embodiment of the disclosure may be isolated.

In any aspect or embodiment of the present disclosure crystalline form O5 of LX9211 Oxalate may be polymorphically pure.

In any aspect or embodiment of the present disclosure, crystalline form O5 of LX9211 Oxalate may be anhydrous.

Crystalline Form O5 of LX9211 Oxalate may be characterized by each of the above characteristics alone/or by all possible combinations, e.g., an XRPD pattern having peaks at 6.3, 7.2, 8.2, 9.1, 11.3, 17.2, 18.2 and 25.3 degrees 2-theta±0.2 degrees 2-theta; and an X-ray powder diffraction pattern substantially as depicted in FIG. 15.

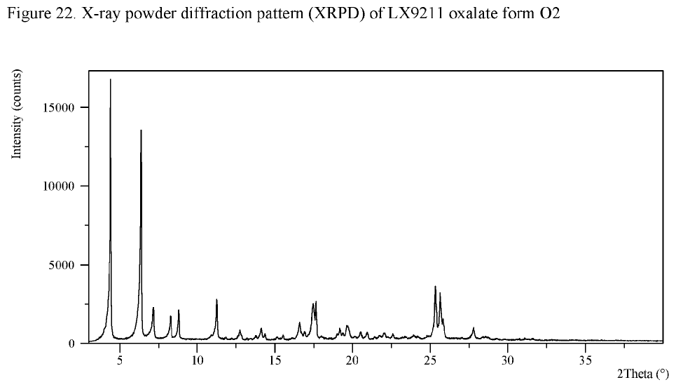

Alternatively, crystalline form O2 of LX9211 Oxalate may be characterized by data selected from one or more of the following: an X-ray powder diffraction pattern substantially as depicted in FIG. 22; an X-ray powder diffraction pattern having peaks at 4.4, 6.3, 7.2, 8.2, 8.8, 11.3, 14.1 and 25.3 degrees 2-theta±0.2 degrees 2-theta. Crystalline form O2 of LX9211 Oxalate may be further characterized an X-ray powder diffraction pattern having peaks at 4.4, 6.3, 7.2, 8.2, 8.8, 11.3, 14.1 and 25.3 degrees 2-theta±0.2 degrees 2-theta and also having any one, two or three additional peaks selected from 16.6, 19.2 and 25.6 degrees 2-theta±0.2 degrees 2-theta. Crystalline form O2 of LX9211 Oxalate may be alternatively characterized by an X-ray powder diffraction pattern having peaks at 4.4, 6.3, 7.2, 8.2, 8.8, 11.3, 14.1, 16.6, 19.2, 25.3 and 25.6 degrees 2-theta±0.2 degrees 2-theta.

Crystalline form O2 of LX9211 Oxalate according to any aspect or embodiment as described herein may be isolated. Particularly, crystalline form O2 of LX9211 Oxalate according to any aspect or embodiment of the disclosure may be isolated.

In any aspect or embodiment of the present disclosure crystalline form O2 of LX9211 Oxalate may be polymorphically pure.

In any aspect or embodiment of the present disclosure, crystalline form O2 of LX9211 Oxalate may be hydrated.

Crystalline form O2 of LX9211 Oxalate may be characterized by each of the above characteristics alone/or by all possible combinations, e.g., an XRPD pattern having peaks at 4.4, 6.3, 7.2, 8.2, 8.8, 11.3, 14.1 and 25.3 degrees 2-theta±0.2 degrees 2-theta; and an X-ray powder diffraction pattern substantially as depicted in FIG. 22.

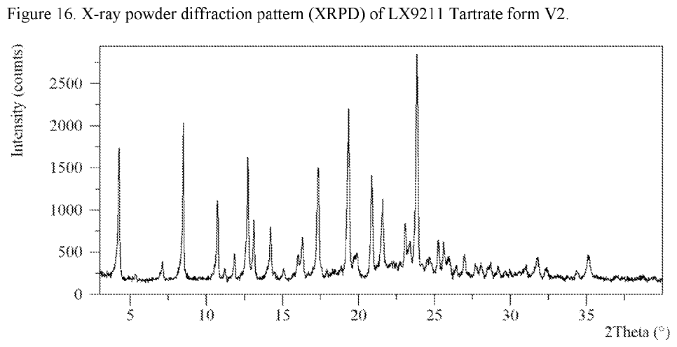

The present disclosure includes a crystalline polymorph LX9211 Tartrate designated Form V2. The crystalline Form V2 of LX9211 Tartrate may be characterized by data selected from one or more of the following: an X-ray powder diffraction pattern substantially as depicted in FIG. 16; an X-ray powder diffraction pattern having peaks at 4.3, 8.5 and 10.7 degrees 2-theta±0.2 degrees 2-theta; and combinations of these data.

Crystalline Form V2 of LX9211 Tartrate may be further characterized by an X-ray powder diffraction pattern having peaks at 4.3, 8.5 and 10.7 degrees 2-theta±0.2 degrees 2-theta, and also having any one or both additional peaks selected from 14.2 and 21.6 degrees 2-theta±0.2 degrees 2-theta.

Crystalline Form V2 of LX9211 Tartrate may be alternatively characterized by an X-ray powder diffraction pattern having peaks at 4.3, 8.5, 10.7, 14.2 and 21.6 degrees 2-theta±0.2 degrees 2-theta.

Crystalline Form V2 of LX9211 Tartrate may be further characterized by an X-ray powder diffraction pattern having peaks at 4.3, 8.5, 10.7, 14.2 and 21.6 degrees 2-theta±0.2 degrees 2-theta, and also having any one, two, three, four, and/or five additional peaks selected from 11.9, 17.3, 19.4, 20.9 and 23.8 degrees 2-theta±0.2 degrees 2-theta.

Crystalline Form V2 of LX9211 Tartrate may be alternatively characterized by an X-ray powder diffraction pattern having peaks at 4.3, 8.5, 10.7, 11.9, 14.2, 17.3, 19.4, 20.9, 21.6, and 23.8 degrees 2-theta±0.2 degrees 2-theta.

In any aspect or embodiment of the present disclosure, crystalline Form V2 of LX9211 Tartrate is isolated. Particularly, crystalline Form V2 of LX9211 Tartrate according to any aspect or embodiment of the disclosure may be isolated.

In any aspect or embodiment of the present disclosure crystalline Form V2 of LX9211 Tartrate may be polymorphically pure.

In any aspect or embodiment of the present disclosure, crystalline Form V2 of LX9211 Tartrate may be anhydrous.

Crystalline Form V2 of LX9211 Tartrate may be characterized by each of the above characteristics alone/or by all possible combinations, e.g., an XRPD pattern having peaks at 4.3, 8.5, 10.7, 14.2 and 21.6 degrees 2-theta±0.2 degrees 2-theta; an XRPD pattern as depicted in FIG. 16, and combinations thereof.

The present disclosure relates to LX9211 dihydrogen phosphate: Urea (or LX9211 phosphoric acid: Urea). Crystalline LX9211 dihydrogen phosphate: Urea (or LX9211 phosphoric acid: Urea) may be a co-crystal of LX9211 dihydrogen phosphate and Urea. Alternatively, crystalline LX9211 dihydrogen phosphate: Urea (or LX9211 phosphoric acid: Urea) may be a salt. Particularly, the present disclosure includes a crystalline polymorph of LX9211 dihydrogen phosphate Urea salt or LX9211 dihydrogen phosphate Urea cocrystal designated Form U1. More particularly, LX9211 dihydrogen phosphate: Urea may be a co-crystal.

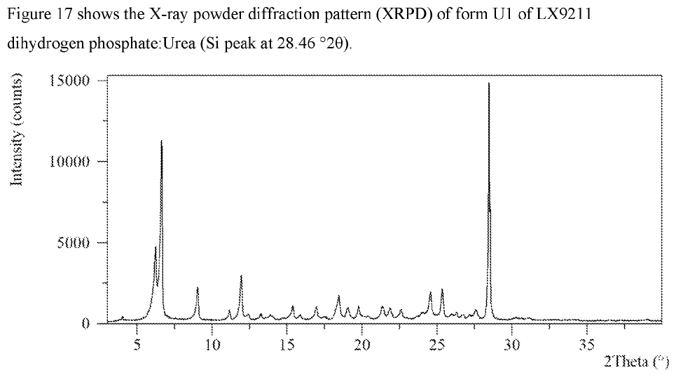

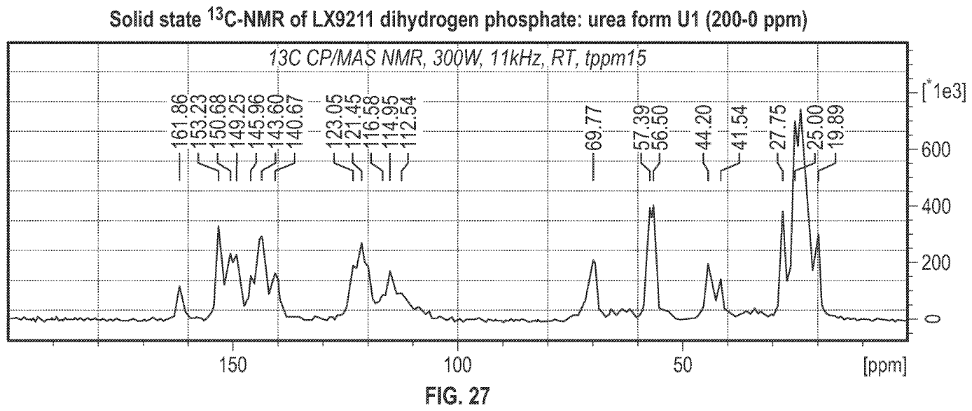

The present disclosure includes a crystalline polymorph LX9211 dihydrogen phosphate: Urea (or LX9211 phosphoric acid: Urea) designated form U1. The crystalline Form U1 of LX9211 dihydrogen phosphate: Urea (or LX9211 phosphoric acid: Urea) may be characterized by data selected from one or more of the following: an X-ray powder diffraction pattern substantially as depicted in FIG. 17; an X-ray powder diffraction pattern having peaks at 6.6, 9.0 and 18.4 degrees 2-theta±0.2 degrees 2-theta; a solid state ¹³C NMR spectrum having characteristic peaks at 153.2, 143.6, 69.8 and 27.8 ppm±0.2 ppm; A solid state ¹³C NMR spectrum having the following chemical shift absolute differences from reference peak at 19.9 ppm±0.2 ppm: 133.3, 123.7, 49.9 and 7.9 ppm±0.1 ppm a solid state ¹³C NMR spectrum substantially as depicted in FIG. 27; and combinations of these data.

Crystalline Form U1 of LX9211 dihydrogen phosphate: Urea (or LX9211 phosphoric acid: Urea) may be further characterized by an X-ray powder diffraction pattern having peaks at 6.6, 9.0 and 18.4 degrees 2-theta±0.2 degrees 2-theta, and also having any one or both additional peaks selected from 11.1 and 16.9 degrees 2-theta±0.2 degrees 2-theta.

Crystalline Form U1 of LX9211 dihydrogen phosphate: Urea (or LX9211 phosphoric acid: Urea) may be alternatively characterized by an X-ray powder diffraction pattern having peaks at 6.6, 9.0, 11.1, 16.9 and 18.4 degrees 2-theta±0.2 degrees 2-theta.

Crystalline Form U1 of LX9211 dihydrogen phosphate: Urea (or LX9211 phosphoric acid: Urea) may be further characterized by an X-ray powder diffraction pattern having peaks at 6.6, 9.0, 11.1, 16.9 and 18.4 degrees 2-theta±0.2 degrees 2-theta, and also having any one, two, three, four, and/or five additional peaks selected from 6.2, 15.4, 19.8, 21.3 and 25.3 degrees 2-theta±0.2 degrees 2-theta.

Crystalline Form U1 of LX9211 dihydrogen phosphate: Urea (or LX9211 phosphoric acid: Urea) may be alternatively characterized by an X-ray powder diffraction pattern having peaks at 6.2, 6.6, 9.0, 11.1, 15.4, 16.9, 18.4, 19.8, 21.3 and 25.3 degrees 2-theta±0.2 degrees 2-theta.

In any aspect or embodiment of the present disclosure, crystalline Form U1 of LX9211 dihydrogen phosphate: Urea (LX9211 phosphoric acid: Urea) is isolated. Particularly, crystalline Form U1 of LX9211 dihydrogen phosphate: Urea (or LX9211 phosphoric acid: Urea) according to any aspect or embodiment of the disclosure may be isolated.

In any aspect or embodiment of the present disclosure crystalline Form U1 of LX9211 dihydrogen phosphate: Urea (or LX9211 phosphoric acid: Urea) may be chemically pure.

In any aspect or embodiment of the present disclosure crystalline Form U1 of LX9211 dihydrogen phosphate: Urea (or LX9211 phosphoric acid: Urea) may be polymorphically pure.

In any aspect or embodiment of the present disclosure, crystalline Form U1 of LX9211 dihydrogen phosphate: Urea (or LX9211 phosphoric acid: Urea) may be anhydrous.

Crystalline Form U1 of LX9211 dihydrogen phosphate: Urea (or LX9211 phosphoric acid: Urea) may be characterized by each of the above characteristics alone/or by all possible combinations, e.g., an XRPD pattern having peaks at 6.6, 9.0, 11.1, 16.9 and 18.4 degrees 2-theta±0.2 degrees 2-theta; an XRPD pattern as depicted in FIG. 17, and combinations thereof.

Crystalline Form U1 of LX9211 dihydrogen phosphate: Urea according to any aspect or embodiment of the present disclosure may be advantageously exhibit improved solubility.

Form U1 of LX9211 dihydrogen phosphate: Urea as described in any aspect or embodiment of the present disclosure, may be prepared by slurrying LX9211 dihydrogen phosphate, preferably form S3 of LX9211 dihydrogen phosphate, and urea in acetonitrile, preferably anhydrous acetonitrile. The starting LX9211 dihydrogen phosphate may be any other form of LX9211 dihydrogen phosphate, but is preferably Form S3 as described in any aspect or embodiment of the present disclosure. Preferably, the slurrying comprises stirring LX9211 dihydrogen phosphate in anhydrous acetonitrile. The acetonitrile may be used in an amount of: about 10 ml to about 60 ml, about 15 ml to about 50 ml, about 20 ml to about 45 ml, about 25 ml to about 40 ml, about 30 ml to about 38 ml, about 32 ml to about 36 ml, or about 34 ml, per gram of LX9211 dihydrogen phosphate. The urea may be used in an amount of: about 1.5 mole equivalents to about 4 mole equivalents, about 1.6 mole equivalents to about 3.0 mole equivalents, about 1.8 mole equivalents to about 2.5 mole equivalents, about 1.9 mole equivalents to about 2.1 mole equivalents or about 2.0 mole equivalents. In any aspect or embodiment of the process, the stirring may be carried out at a temperature of: about 40° C. to about 80° C., about 50° C. to about 70° C., or about 60° C. The stirring may be carried out for a period of: about 30 minutes to about 8 hours, about 1 hour to about 4 hours, about 1 hour to about 3 hours, or about 2 hours. The mixture may be cooled, or allowed to cool to about room temperature. The mixture may be further stirred at room temperature for any suitable time to prepare form U1 of LX9211 dihydrogen phosphate. Preferably, the stirring may be for a period of: about 1 hour to about 5 days, about 2 hours to about 4 days, or about 3 days. The product may be isolated by any suitable procedure, such as decantation, centrifugation or filtration, preferably by filtration. The resulting solid may be dried, optionally at: about 20° C. to about 70° C., about 30° C. to about 65° C., about 40° C. to about 60° C., 45° C. to about 55° C., or about 50° C. The drying may be carried out for any suitable period of time, particularly about 20 minutes to about 6 hours, about 45 minutes to about 5 hours, about 1 hour to about 4.5 hours, about 2.5 hours to about 4 hours, or about 3 hours, optionally under vacuum.

The present disclosure further encompasses a product obtainable by any of the above-described processes.

The process may further comprise combining the Form U1 of LX9211 dihydrogen phosphate: Urea with at least one pharmaceutically acceptable excipient to form a pharmaceutical composition

The present disclosure relates to LX9211 dihydrogen phosphate: L-(+)-Tartaric acid (or LX9211 phosphoric acid: L-(+)-Tartaric acid). Crystalline LX9211 dihydrogen phosphate: L-(+)-Tartaric acid (or LX9211 phosphoric acid: L-(+)-Tartaric acid) may be a co-crystal of LX9211 dihydrogen phosphate and L-(+)-Tartaric acid. Alternatively, crystalline LX9211 dihydrogen phosphate: L-(+)-Tartaric acid (or LX9211 phosphoric acid: L-(+)-Tartaric acid) may be a salt.

Particularly, the present disclosure includes a crystalline polymorph of LX9211 dihydrogen phosphate L-(+)-Tartaric acid (or LX9211 phosphoric acid: L-(+)-Tartaric acid) salt or LX9211 dihydrogen phosphate L-(+)-Tartaric acid (or LX9211 phosphoric acid: L-(+)-Tartaric acid) cocrystal designated Form V3.

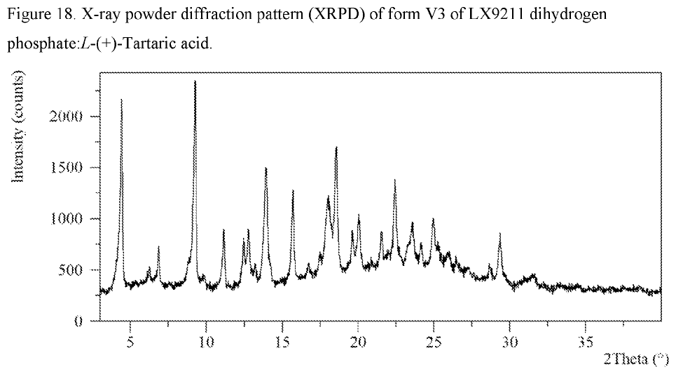

The present disclosure includes a crystalline polymorph LX9211 dihydrogen phosphate: L-(+)-Tartaric acid (or LX9211 phosphoric acid: L-(+)-Tartaric acid) designated form V3. The crystalline Form V3 of LX9211 dihydrogen phosphate: L-(+)-Tartaric acid (or LX9211 phosphoric acid: L-(+)-Tartaric acid) may be characterized by data selected from one or more of the following: an X-ray powder diffraction pattern substantially as depicted in FIG. 18; an X-ray powder diffraction pattern having peaks at 9.3, 11.2 and 18.6 degrees 2-theta±0.2 degrees 2-theta; and combinations of these data.

Crystalline Form V3 of LX9211 dihydrogen phosphate: L-(+)-Tartaric acid (or LX9211 phosphoric acid: L-(+)-Tartaric acid) may be further characterized by an X-ray powder diffraction pattern having peaks at 9.3, 11.2 and 18.6 degrees 2-theta±0.2 degrees 2-theta, and also having any one or both additional peaks selected from 12.8 and 13.9 degrees 2-theta±0.2 degrees 2-theta.

Crystalline Form V3 of LX9211 dihydrogen phosphate: L-(+)-Tartaric acid (or LX9211 phosphoric acid: L-(+)-Tartaric acid) may be alternatively characterized by an X-ray powder diffraction pattern having peaks at 9.3, 11.2, 12.8, 13.9 and 18.6 degrees 2-theta±0.2 degrees 2-theta.

Crystalline Form V3 of LX9211 dihydrogen phosphate: L-(+)-Tartaric acid (or LX9211 phosphoric acid: L-(+)-Tartaric acid) may be further characterized by an X-ray powder diffraction pattern having peaks at 9.3, 11.2, 12.8 and 13.9 and 18.6 degrees 2-theta±0.2 degrees 2-theta, and also having any one, two, three, four, and/or five additional peaks selected from 4.4, 12.5, 15.7, 19.6 and 25.0 degrees 2-theta±0.2 degrees 2-theta.

Crystalline Form V3 of LX9211 dihydrogen phosphate: L-(+)-Tartaric acid (or LX9211 phosphoric acid: L-(+)-Tartaric acid) may be alternatively characterized by an X-ray powder diffraction pattern having peaks at 4.4, 9.3, 11.2, 12.5, 12.8, 13.9, 15.7, 18.6, 19.6 and 25.0 degrees 2-theta±0.2 degrees 2-theta.

In any aspect or embodiment of the present disclosure, crystalline Form V3 of LX9211 dihydrogen phosphate: L-(+)-Tartaric acid (or LX9211 phosphoric acid: L-(+)-Tartaric acid) is isolated. Particularly, crystalline Form V3 of LX9211 dihydrogen phosphate: L-(+)-Tartaric acid (or LX9211 phosphoric acid: L-(+)-Tartaric acid) according to any aspect or embodiment of the disclosure may be isolated.

In any embodiment of the present disclosure crystalline Form V3 of LX9211 dihydrogen phosphate: L-(+)-Tartaric acid (or LX9211 phosphoric acid: L-(+)-Tartaric acid) may be polymorphically pure.

Crystalline Form V3 of LX9211 dihydrogen phosphate: L-(+)-Tartaric acid (or LX9211 phosphoric acid: L-(+)-Tartaric acid) may be characterized by each of the above characteristics alone/or by all possible combinations, e.g., an XRPD pattern having peaks at 9.3, 11.2, 12.8 and 13.9 and 18.6 degrees 2-theta±0.2 degrees 2-theta; an XRPD pattern as depicted in FIG. 18, and combinations thereof.

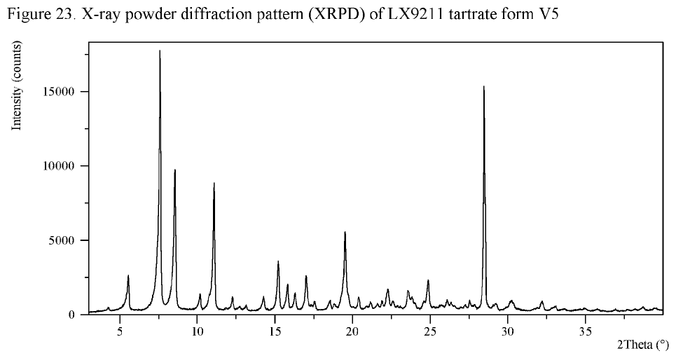

The present disclosure includes a crystalline polymorph LX9211 tartrate designated Form V5. The crystalline Form V5 of LX9211 tartrate may be characterized by data selected from one or more of the following: an X-ray powder diffraction pattern substantially as depicted in FIG. 23; an X-ray powder diffraction pattern having peaks at 5.5, 7.6 and 11.1 degrees 2-theta±0.2 degrees 2-theta; and combinations of these data.

Crystalline Form V5 of LX9211 tartrate may be further characterized by an X-ray powder diffraction pattern having peaks at 5.5, 7.6 and 11.1 degrees 2-theta±0.2 degrees 2-theta, and also having any one or both additional peaks selected from 10.2 and 15.2 degrees 2-theta±0.2 degrees 2-theta.

The crystalline Form V5 of LX9211 tartrate may be alternatively characterized by an X-ray powder diffraction pattern having peaks at 5.5, 7.6, 10.2, 11.1 and 15.2±0.2 degrees 2-theta; an XRPD pattern as depicted in FIG. 23 and combinations of these data.

Crystalline Form V5 of LX9211 tartrate may be further characterized by an X-ray powder diffraction pattern having peaks at 5.5, 7.6, 10.2, 11.1 and 15.2±0.2 degrees 2-theta±0.2 degrees 2-theta, and also having any one, two, three, four or five additional peaks selected from 12.2, 15.8, 17.0, 22.3 and 24.8±0.2 degrees 2-theta±0.2 degrees 2-theta.

Crystalline Form V5 of LX9211 tartrate may be alternatively characterized by an X-ray powder diffraction pattern having peaks at 5.5, 7.6, 10.2, 11.1, 12.2, 15.2, 15.8, 17.0, 22.3 and 24.8 degrees 2-theta±0.2 degrees 2-theta.

In any aspect or embodiment of the present disclosure, crystalline Form V5 of LX9211 tartrate is isolated. Particularly, crystalline Form V5 of LX9211 tartrate according to any aspect or embodiment of the disclosure may be isolated.

In any aspect or embodiment crystalline Form V5 of LX9211 tartrate may be polymorphically pure.

Crystalline Form V5 of LX9211 tartrate may be characterized by each of the above characteristics alone/or by all possible combinations, e.g., an XRPD pattern having peaks at 5.5, 7.6, 10.2, 11.1 and 15.2 degrees 2-theta±0.2 degrees 2-theta; an XRPD pattern as depicted in FIG. 23, and combinations thereof.

The present disclosure relates to LX9211 dihydrogen phosphate: Oxalic acid (or LX9211 phosphoric acid: L-(+)-Oxalic acid). Crystalline LX9211 dihydrogen phosphate: Oxalic acid (or LX9211 phosphoric acid: L-(+)-Oxalic acid) may be a co-crystal of LX9211 dihydrogen phosphate and Oxalic acid. Alternatively, crystalline LX9211 dihydrogen phosphate: Oxalic acid (or LX9211 phosphoric acid: L-(+)-Oxalic acid) may be a salt. Particularly, the present disclosure includes a crystalline polymorph of LX9211 dihydrogen phosphate Oxalic acid (or LX9211 phosphoric acid: L-(+)-Oxalic acid) salt or LX9211 dihydrogen phosphate Oxalic acid (or LX9211 phosphoric acid: L-(+)-Oxalic acid) cocrystal designated Form O4.

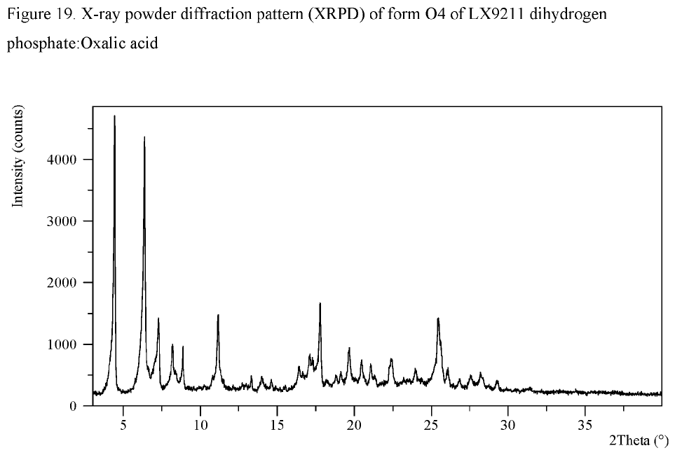

The present disclosure includes a crystalline polymorph LX9211 dihydrogen phosphate: Oxalic acid (or LX9211 phosphoric acid: L-(+)-Oxalic acid) designated form O4. The crystalline Form O4 of LX9211 dihydrogen phosphate: Oxalic acid (or LX9211 phosphoric acid: L-(+)-Oxalic acid) may be characterized by data selected from one or more of the following: an X-ray powder diffraction pattern substantially as depicted in FIG. 19; an X-ray powder diffraction pattern having peaks at 6.4, 11.2 and 19.7 degrees 2-theta±0.2 degrees 2-theta; and combinations of these data.

Crystalline Form O4 of LX9211 dihydrogen phosphate: Oxalic acid (or LX9211 phosphoric acid: L-(+)-Oxalic acid) may be further characterized by an X-ray powder diffraction pattern having peaks at 6.4, 11.2 and 19.7 degrees 2-theta±0.2 degrees 2-theta, and also having any one or both additional peaks selected from 7.3 and 8.2 degrees 2-theta±0.2 degrees 2-theta.

Crystalline Form O4 of LX9211 dihydrogen phosphate: Oxalic acid (or LX9211 phosphoric acid: L-(+)-Oxalic acid) may be alternatively characterized by an X-ray powder diffraction pattern having peaks at 6.4, 7.3, 8.2, 11.2 and 19.7 degrees 2-theta±0.2 degrees 2-theta.

Crystalline Form O4 of LX9211 dihydrogen phosphate: Oxalic acid or LX9211 phosphoric acid: L-(+)-Oxalic acid) may be further characterized by an X-ray powder diffraction pattern having peaks at 6.4, 7.3, 8.2, 11.2 and 19.7 degrees 2-theta±0.2 degrees 2-theta, and also having any one, two, three, four, and/or five additional peaks selected from 8.9, 16.4, 20.5, 22.4 and 25.4 degrees 2-theta±0.2 degrees 2-theta.

Crystalline Form O4 of LX9211 dihydrogen phosphate: Oxalic acid (or LX9211 phosphoric acid: L-(+)-Oxalic acid) may be alternatively characterized by an X-ray powder diffraction pattern having peaks at 6.4, 7.3, 8.2, 8.9, 11.2, 16.4, 19.7, 20.5, 22.4 and 25.4 degrees 2-theta±0.2 degrees 2-theta.

In any aspect or embodiment of the present disclosure, crystalline Form O4 of LX9211 dihydrogen phosphate: Oxalic acid (or LX9211 phosphoric acid: L-(+)-Oxalic acid) is isolated. Particularly, crystalline Form O4 of LX9211 dihydrogen phosphate: Oxalic acid (or LX9211 phosphoric acid: L-(+)-Oxalic acid) according to any aspect or embodiment of the disclosure may be isolated.

In any aspect or embodiment of the present disclosure crystalline Form O4 of LX9211 dihydrogen phosphate: Oxalic acid (or LX9211 phosphoric acid: L-(+)-Oxalic acid) may be polymorphically pure.

Crystalline Form O4 of LX9211 dihydrogen phosphate: Oxalic acid (or LX9211 phosphoric acid: L-(+)-Oxalic acid) may be characterized by each of the above characteristics alone/or by all possible combinations, e.g., an XRPD pattern having peaks at 6.4, 7.3, 8.2, 11.2 and 19.7 degrees 2-theta±0.2 degrees 2-theta; an XRPD pattern as depicted in FIG. 19, and combinations thereof. The above crystalline polymorphs can be used to prepare other crystalline polymorphs of LX9211, LX9211 salts and their solid state forms.

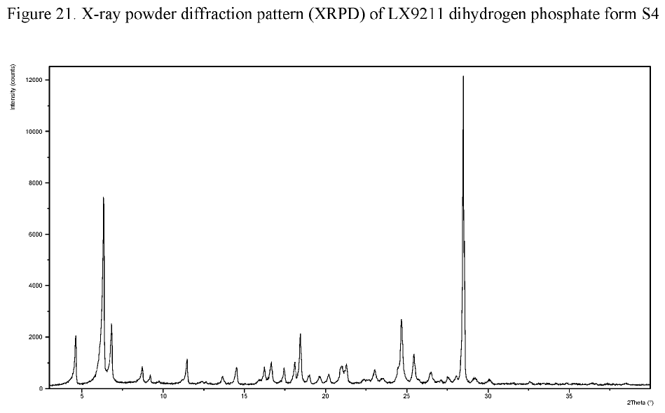

The present disclosure includes a crystalline polymorph LX9211 dihydrogen phosphate designated Form S4. The crystalline Form S4 of LX9211 dihydrogen phosphate may be characterized by data selected from one or more of the following: an X-ray powder diffraction pattern substantially as depicted in FIG. 21; an X-ray powder diffraction pattern having peaks at 6.3, 18.4 and 25.4 degrees 2-theta±0.2 degrees 2-theta; and combinations of these data.

Crystalline Form S4 of LX9211 dihydrogen phosphate may be further characterized by an X-ray powder diffraction pattern having peaks at 6.3, 18.4 and 25.4 degrees 2-theta±0.2 degrees 2-theta, and also having any one or both additional peaks selected from 14.5 and 17.4 degrees 2-theta±0.2 degrees 2-theta.

The crystalline Form S4 of LX9211 dihydrogen phosphate may be alternatively characterized by an X-ray powder diffraction pattern having peaks at 6.3, 14.5, 17.4, 18.4 and 25.4±0.2 degrees 2-theta; an XRPD pattern as depicted in FIG. 21 and combinations of these data.

Crystalline Form S4 of LX9211 dihydrogen phosphate may be further characterized by an X-ray powder diffraction pattern having peaks at 6.3, 14.5, 17.4, 18.4 and 25.4±0.2 degrees 2-theta±0.2 degrees 2-theta, and also having any one, two, three, four or five additional peaks selected from 4.6, 6.8, 16.2, 21.3 and 26.5±0.2 degrees 2-theta±0.2 degrees 2-theta.

Crystalline Form S4 of LX9211 dihydrogen phosphate may be alternatively characterized by an X-ray powder diffraction pattern having peaks at 4.6, 6.3, 6.8, 14.5, 16.2, 17.4, 18.4, 21.3, 25.4 and 26.5 degrees 2-theta±0.2 degrees 2-theta.

In any aspect or embodiment of the present disclosure, crystalline Form S4 of LX9211 dihydrogen phosphate is isolated. Particularly, crystalline Form S4 of LX9211 dihydrogen phosphate according to any aspect or embodiment of the disclosure may be isolated.

In any aspect or embodiment crystalline Form S4 of LX9211 dihydrogen phosphate may be polymorphically pure.

Crystalline Form S4 of LX9211 dihydrogen phosphate may be characterized by each of the above characteristics alone/or by all possible combinations, e.g., an XRPD pattern having peaks at 6.3, 14.5, 17.4, 18.4 and 25.4 degrees 2-theta±0.2 degrees 2-theta; an XRPD pattern as depicted in FIG. 21, and combinations thereof.

The present disclosure encompasses a process for preparing other solid state forms of LX9211, LX9211 salts and solid state forms, or cocrystals thereof. The process includes preparing any one of the solid state form of LX9211 or the salts, or cocrystals thereof by the processes of the present disclosure, and converting that form to a different form of LX9211 or the salts, or cocrystals thereof. For example, the process may include preparing any one of the solid state forms of LX9211 salts, or cocrystals of the present disclosure by the processes of the present disclosure, and converting it to said other form of LX9211 or LX9211 salt or LX9211 cocrystal. The conversion can be done, for example, by a process comprising basifying any one of the above described forms of LX9211 salts and reacting the obtained LX9211 with an appropriate acid, to obtain the corresponding salt. Alternatively, the conversion can be done by salt switching, i.e., reacting any one of the forms of the LX9211 salt of the present disclosure with an acid having a pKa which is lower than that of the acid of the original salt.

Any of the above described crystalline forms of LX9211, salts or co-crystals thereof described above may be used for purification of LX9211. Thus, the present disclosure encompasses the use of any of the above described crystalline forms of LX9211, salts or co-crystals thereof described herein, as an intermediate for the purification of LX9211. For example, the purification of LX9211 may be carried out by preparing any the crystalline forms of LX9211, salts or co-crystals thereof as described herein, using any of the processes described herein, and basifying or removing the co-crystal former, to obtain purified LX9211. Thus, for example, the LX9211 starting material to be purified may be converted to any the crystalline forms of LX9211, salts or co-crystals thereof as described herein, and converted back to LX9211 by removal of the salt or cocrystal former.

The present disclosure provides the above described crystalline polymorphs of LX9211 for use in the preparation of pharmaceutical compositions comprising LX9211 and/or solid state forms thereof.

The present disclosure also encompasses the use of crystalline polymorphs of LX9211 of the present disclosure for the preparation of pharmaceutical compositions of crystalline polymorph LX9211 and/or solid state forms thereof.

The present disclosure includes processes for preparing the above mentioned pharmaceutical compositions. The processes include combining any one or a combination of the crystalline polymorph of LX9211 of the present disclosure with at least one pharmaceutically acceptable excipient.

Pharmaceutical combinations or formulations of the present disclosure contain any one or a combination of the solid state form of LX9211 of the present disclosure. In addition to the active ingredient, the pharmaceutical formulations of the present disclosure can contain one or more excipients. Excipients are added to the formulation for a variety of purposes.

Diluents increase the bulk of a solid pharmaceutical composition, and can make a pharmaceutical dosage form containing the composition easier for the patient and caregiver to handle. Diluents for solid compositions include, for example, microcrystalline cellulose (e.g., Avicel®), microfine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g., Eudragit®), potassium chloride, powdered cellulose, sodium chloride, sorbitol, and talc.

Solid pharmaceutical compositions that are compacted into a dosage form, such as a tablet, can include excipients whose functions include helping to bind the active ingredient and other excipients together after compression. Binders for solid pharmaceutical compositions include acacia, alginic acid, carbomer (e.g. carbopol), carboxymethylcellulose sodium, dextrin, ethyl cellulose, gelatin, guar gum, hydrogenated vegetable oil, hydroxyethyl cellulose, hydroxypropyl cellulose (e.g. Klucel®), hydroxypropyl methyl cellulose (e.g. Methocel®), liquid glucose, magnesium aluminum silicate, maltodextrin, methylcellulose, polymethacrylates, povidone (e.g. Kollidon®, Plasdone®), pregelatinized starch, sodium alginate, and starch.

The dissolution rate of a compacted solid pharmaceutical composition in the patient's stomach can be increased by the addition of a disintegrant to the composition. Disintegrants include alginic acid, carboxymethylcellulose calcium, carboxymethylcellulose sodium (e.g., Ac-Di-Sol®, Primellose®), colloidal silicon dioxide, croscarmellose sodium, crospovidone (e.g., Kollidon®, Polyplasdone®), guar gum, magnesium aluminum silicate, methyl cellulose, microcrystalline cellulose, polacrilin potassium, powdered cellulose, pregelatinized starch, sodium alginate, sodium starch glycolate (e.g., Explotab®), and starch.

Glidants can be added to improve the flowability of a non-compacted solid composition and to improve the accuracy of dosing. Excipients that can function as glidants include colloidal silicon dioxide, magnesium trisilicate, powdered cellulose, starch, talc, and tribasic calcium phosphate.

When a dosage form such as a tablet is made by the compaction of a powdered composition, the composition is subjected to pressure from a punch and dye. Some excipients and active ingredients have a tendency to adhere to the surfaces of the punch and dye, which can cause the product to have pitting and other surface irregularities. A lubricant can be added to the composition to reduce adhesion and ease the release of the product from the dye. Lubricants include magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc, and zinc stearate.

Flavoring agents and flavor enhancers make the dosage form more palatable to the patient. Common flavoring agents and flavor enhancers for pharmaceutical products that can be included in the composition of the present disclosure include maltol, vanillin, ethyl vanillin, menthol, citric acid, fumaric acid, ethyl maltol, and tartaric acid.

Solid and liquid compositions can also be dyed using any pharmaceutically acceptable colorant to improve their appearance and/or facilitate patient identification of the product and unit dosage level.

In liquid pharmaceutical compositions of the present invention, LX9211 and any other solid excipients can be dissolved or suspended in a liquid carrier such as water, vegetable oil, alcohol, polyethylene glycol, propylene glycol, or glycerin.

Liquid pharmaceutical compositions can contain emulsifying agents to disperse uniformly throughout the composition an active ingredient or other excipient that is not soluble in the liquid carrier. Emulsifying agents that can be useful in liquid compositions of the present invention include, for example, gelatin, egg yolk, casein, cholesterol, acacia, tragacanth, chondrus, pectin, methyl cellulose, carbomer, cetostearyl alcohol, and cetyl alcohol.

Liquid pharmaceutical compositions of the present invention can also contain a viscosity enhancing agent to improve the mouth-feel of the product and/or coat the lining of the gastrointestinal tract. Such agents include acacia, alginic acid bentonite, carbomer, carboxymethylcellulose calcium or sodium, cetostearyl alcohol, methyl cellulose, ethylcellulose, gelatin guar gum, hydroxyethyl cellulose, hydroxypropyl cellulose, hydroxypropyl methyl cellulose, maltodextrin, polyvinyl alcohol, povidone, propylene carbonate, propylene glycol alginate, sodium alginate, sodium starch glycolate, starch tragacanth, xanthan gum and combinations thereof.

Sweetening agents such as sorbitol, saccharin, sodium saccharin, sucrose, aspartame, fructose, mannitol, and invert sugar can be added to improve the taste.

Preservatives and chelating agents such as alcohol, sodium benzoate, butylated hydroxyl toluene, butylated hydroxyanisole, and ethylenediamine tetraacetic acid can be added at levels safe for ingestion to improve storage stability.

According to the present disclosure, a liquid composition can also contain a buffer such as gluconic acid, lactic acid, citric acid, or acetic acid, sodium gluconate, sodium lactate, sodium citrate, or sodium acetate. Selection of excipients and the amounts used can be readily determined by the formulation scientist based upon experience and consideration of standard procedures and reference works in the field.

The solid compositions of the present disclosure include powders, granulates, aggregates, and compacted compositions. The dosages include dosages suitable for oral, buccal, rectal, parenteral (including subcutaneous, intramuscular, and intravenous), inhalant, and ophthalmic administration. Although the most suitable administration in any given case will depend on the nature and severity of the condition being treated, in embodiments the route of administration is oral. The dosages can be conveniently presented in unit dosage form and prepared by any of the methods well-known in the pharmaceutical arts.

Dosage forms include solid dosage forms like tablets, powders, capsules, suppositories, sachets, troches, and lozenges, as well as liquid syrups, suspensions, and elixirs.

The dosage form of the present disclosure can be a capsule containing the composition, such as a powdered or granulated solid composition of the disclosure, within either a hard or soft shell. The shell can be made from gelatin and optionally contain a plasticizer such as glycerin and/or sorbitol, an opacifying agent and/or colorant.

The active ingredient and excipients can be formulated into compositions and dosage forms according to methods known in the art.

A composition for tableting or capsule filling can be prepared by wet granulation. In wet granulation, some or all of the active ingredients and excipients in powder form are blended and then further mixed in the presence of a liquid, typically water that causes the powders to clump into granules. The granulate is screened and/or milled, dried, and then screened and/or milled to the desired particle size. The granulate can then be tableted, or other excipients can be added prior to tableting, such as a glidant and/or a lubricant.

A tableting composition can be prepared conventionally by dry blending. For example, the blended composition of the actives and excipients can be compacted into a slug or a sheet and then comminuted into compacted granules. The compacted granules can subsequently be compressed into a tablet.

As an alternative to dry granulation, a blended composition can be compressed directly into a compacted dosage form using direct compression techniques. Direct compression produces a more uniform tablet without granules. Excipients that are particularly well suited for direct compression tableting include microcrystalline cellulose, spray dried lactose, dicalcium phosphate dihydrate, and colloidal silica. The proper use of these and other excipients in direct compression tableting is known to those in the art with experience and skill in particular formulation challenges of direct compression tableting.

A capsule filling of the present disclosure can include any of the aforementioned blends and granulates that were described with reference to tableting, but they are not subjected to a final tableting step.

A pharmaceutical formulation of LX9211 can be administered. LX9211 may be formulated for administration to a mammal, in embodiments to a human, by injection. LX9211 can be formulated, for example, as a viscous liquid solution or suspension, such as a clear solution, for injection. The formulation can contain one or more solvents. A suitable solvent can be selected by considering the solvent's physical and chemical stability at various pH levels, viscosity (which would allow for syringeability), fluidity, boiling point, miscibility, and purity. Suitable solvents include alcohol USP, benzyl alcohol NF, benzyl benzoate USP, and Castor oil USP. Additional substances can be added to the formulation such as buffers, solubilizers, and antioxidants, among others. Ansel et al., Pharmaceutical Dosage Forms and Drug Delivery Systems, 7th ed.

The crystalline polymorphs of LX9211 and the pharmaceutical compositions and/or formulations of LX9211 of the present disclosure can be used as medicaments, in embodiments for the treatment of patients with Diabetic Peripheral Neuropathic pain and/or Post-Herpetic Neuralgia.

The present disclosure also provides methods of treating of patients with Diabetic Peripheral Neuropathic pain and/or Post-Herpetic Neuralgia by administering a therapeutically effective amount of any one or a combination of the crystalline polymorphs of LX9211 of the present disclosure, or at least one of the above pharmaceutical compositions and/or formulations, to a subject in need of the treatment.

Having thus described the disclosure with reference to particular preferred embodiments and illustrative examples, those in the art can appreciate modifications to the disclosure as described and illustrated that do not depart from the spirit and scope of the disclosure as disclosed in the specification. The Examples are set forth to aid in understanding the disclosure but are not intended to, and should not be construed to limit its scope in any way.

Further aspects and embodiments of the present disclosure are set out in the numbered clauses below:
1. A crystalline solid which comprises LX9211 and Phosphoric acid.
2. A crystalline solid according to Clause 1 which is a salt or a co-crystal.
3. A crystalline solid according to Clause 1 or Clause 2 which is an acid salt of LX9211.
4. A crystalline solid according to any one of Clauses 1, 2 or 3 wherein the molar ration of LX9211 to Phosphoric acid is 2:1.
5. A crystalline solid according to any one of Clauses 1, 2, 3 or 4 which is a hemihydrogen phosphate salt of LX9211.
6. A crystalline solid according to any one of Clauses 1, 2 or 4 which is a co-crystal of a dihydrogen phosphate salt of LX9211.
7. A crystalline solid according to Clause 6 which is a co-crystal of phosphoric acid salt LX9211 with LX9211.
8. A crystalline solid according to Clause 7 which is a co-crystal of LX9211 dihydrogen phosphate with LX9211.
9. A crystalline solid according to any one of Clauses 1-7, designated Form S5 of LX9211: phosphoric acid, which is characterized by dtat selected from one or more of the following:
   a) an X-ray powder diffraction pattern having peaks at 4.5, 11.9 and 18.1 degrees 2-theta±0.2 degrees 2-theta;
   b) an X-ray powder diffraction pattern substantially as depicted in FIG. 9; and/or
   c) combinations of these data.
10. A Crystalline solid according to Clause 9 characterized by the XRPD pattern having peaks at 4.5, 11.9 and 18.1 degrees 2-theta±0.2 degrees 2-theta and also having any one or both additional peaks selected from 6.9 and 10.0 degrees 2-theta±0.2 degrees 2-theta.
11. A crystalline solid according to Clause 10 characterized by the XRPD pattern having peaks at 4.5, 6.9, 10.0, 11.9 and 18.1 degrees 2-theta±0.2 degrees 2-theta.
12. A Crystalline solid according to Clause 11 characterized by the XRPD pattern having peaks at 4.5, 6.9, 10.0, 11.9 and 18.1 degrees 2-theta±0.2 degrees 2-theta, and also having any one, two, three, four or five additional peaks selected from 12.5, 17.7, 20.1, 25.7 and 27.8 degrees 2-theta±0.2 degrees 2-theta.
13. A crystalline solid according to any one of Clauses 1-11 wherein the form is a hydrate.
14. A crystalline solid according to any one of Clauses 1-13 which contains: no more than about 20%, no more than about 10%, no more than about 5%, no more than about 2%, no more than about 1% or about 0% of any other crystalline forms of LX9211.
15. A crystalline solid according to any one of Clauses 1-13 which contains: no more than about 20%, no more than about 10%, no more than about 5%, no more than about 2%, no more than about 1% or about 0% of amorphous LX9211.

### Powder X-Ray Diffraction (“XRPD”) Method

Sample after being powdered in a mortar and pestle is applied directly on a silicon plate holder. The X-ray powder diffraction pattern was measured with Philips X'Pert PRO X-ray powder diffractometer, equipped with Cu irradiation source=1.54184 \acute{Å} (\acute{Å}ngström), X'Celerator (2.022° 2θ) detector. Scanning parameters: angle range: 3-40 deg., step size 0.0167, time per step 37 s, continuous scan.

The described peak positions were determined with using silicon powder as an internal standard in an admixture with the sample measured.

The position of the silicon (Si) peak was corrected to silicone theoretical peak: 28.45 degrees two theta, and the positions of the measured peaks were corrected, respectively.

### SCXRD Method

A suitable single crystal was selected and mounted on the goniometer of Rigaku XtaLAB Synergy diffractometer equipped with Dualflex source (Cu Kα radiation, λ=1.54184 Å). Diffracted intensities were collected with HyPix detector using @-scans. The crystal was kept at 170 K during data collection. Data were prepared using the CrysAlis program package. The structures were solved with dual space methods using SHELXT. The refinement procedure by full-matrix least-squares methods based on F2 values against all reflections included anisotropic displacement parameters for all non-H atoms. Hydrogen atoms bound to carbon atoms and heteroatoms were placed in geometrically idealized positions and refined by the use of the riding model with Uiso±1.2 Ueq of the connected carbon atom or as ideal CH3 groups with Uiso=1.5 Ueq. All refinements were performed using SHELXL. The SHELX programs operated within the Olex2 suite.

Cell parameters for room-temperature data were refined from powder diffraction data, collected in capillary transmission mode on Panalytical Empyrean diffractometer equipped with Cu Kα focusing optics and PixCel3D detector. Refinement was done using least squares method within JANA2006 software.

### SS-NMR Method

Solid-state NMR spectra of forms S1, S3 and S5 were measured at 16.4 T using a Bruker Avance NEO 700 SB NMR spectrometer (Karlsruhe, Germany, 2021) with 3.2 mm probehead.

Solid-state NMR spectrum of form U1 was measured at 11.7 T using a Bruker Avance III HD 500 US/WB NMR spectrometer (Karlsruhe, Germany, 2013) with a 4 mm probe head.

The ¹³C CP/MAS NMR spectra employing cross-polarization are acquired using the standard cross-polarization pulse scheme at spinning frequency of 11 or 18 kHz. The dipolar decoupling SPINAL64 is applied during the data acquisition. The number of scans is set for the signal-to-noise ratio SINO reaches at least the value ca. 50. The ¹³C scale is referenced to α-glycine (176.03 ppm for ¹³C).

Frictional heating of the spinning samples is compensated by active cooling, and the temperature calibration is performed with Pb(NO₃)₂.

The NMR spectrometer is always completely calibrated and all experimental parameters are carefully optimized prior the recording of the spectra. Magic angle is set using KBr during the standard optimization procedure and homogeneity of magnetic field is optimized using adamantane sample (resulting line-width at half-height Δv½ was less than 3.5 Hz at 250 ms of acquisition time).

### DSC Method

Sample (about 3 mg) was subjected to thermal treatment on DSC Discovery TA instrument in a pin-hole aluminum closed pan. Heating rate was 10° C./minute up to temperature of 300° C., with nitrogen purge of 30 mL/min.

### EXAMPLES

### Preparation of Starting Materials

LX9211 can be prepared according to methods known from the literature, for example according to the disclosure in International Publication No. WO 2015/153720.

### Example 1: Preparation of LX9211 Crystal Form A

50 mg of LX9211 base was dissolved in 1.5 mL of ethyl acetate at room temperature. The obtained solution was kept at 5-10° C., covered with Parafilm® with small holes punched through the film to allow slow evaporation of the solvent. After two days the solvent evaporated and the solid material obtained was analyzed and characterized by X-ray powder diffraction as LX9211 Form A. The XRPD pattern is presented in FIG. 1.

Form A may be prepared according to the above the same procedure using the following solvents:

| Solvent | volume | |
|---|---|---|
| Methanol | 1 | ml |
| Ethanol 96% | 2 | ml |
| Ethanol absolute | 0.3 | ml |
| Diethyl ether | 0.6 | ml |
| Diisopropyl ether | 0.6 | ml |
| Acetone | 1 | ml |
| Chloroform | 0.6 | ml |
| Dichloromethane | 0.6 | ml |

Form A shows a melting endotherm with an onset temperature of about 40-42° C.

### Example 2: Preparation of LX9211 Crystal Form B

### Procedure A

30 mg of LX9211 base was placed in a vial and a 0.3 mL mixture of water with 15% of 1-PrOH was added. Slurry was left to stir at 0-10° C. for 6 days. After 6 days, slurry was filtered off and sample was analysed by XRPD and the XRPD pattern is presented in FIG. 2.

### Procedure B

30 mg of LX9211 base (prepared according to example 1) was placed in a vial and a 0.3 mL mixture of water with 15% of 1-PrOH was added. Slurry was left to stir at 0-10° C. for 6 days. After 6 days, slurry was filtered off and sample was analysed by XRPD and identified as Form B.

### Procedure C

30 mg of LX9211 base was placed in a vial and a 0.3 mL mixture of water with 15% of MeOH was added. Slurry was left to stir at 0-10° C. for 6 days. After 6 days, slurry was filtered off and sample was analysed by XRPD and identified as Form B.

### Procedure D

30 mg of LX9211 base (prepared according to example 1) was placed in a vial and a 0.3 mL mixture of water with 15% of MeOH was added. Slurry was left to stir at 0-10° C. for 6 days. After 6 days, slurry was filtered off and sample was analysed by XRPD and identified as Form B.

### Procedure E

LX9211 base form A (approximately 30 mg) was put in an opened Eppendorf tube and exposed to water atmosphere in a chamber at 40° C. After 1 month the sample was analyzed by XRPD. LX9211 base form B was obtained and the XRPD pattern is presented in FIG. 2A.

### Example 3: Preparation of LX9211 Dihydrogen Phosphate Form S1

### Procedure A

LX9211 (1 gram) was dissolved in 2-propanol (5 mL) (concentration 200 g/L) at room temperature (25° C.) to obtain a solution. Phosphoric acid (85%) was added dropwise (0.4057 ml, 2.2 eq). To the obtained solution water was added dropwise (20 ml) after which the flask was placed in ice bath and left stirring for 1 day. Crystallization occurred. The solid was isolated by vacuum filtration. The solid was dried in oven at 30° C. for 2 hours. The obtained solid was analyzed by XRD and characterized as LX9211 dihydrogen phosphate form S1 and the XRD pattern is presented in FIG. 3.

### Procedure B

LX9211 dihydrogen phosphate form S1 (approximately 30 mg, prepared according to example 3, procedure A) was put in Petri dish and opened placed in chamber with 40% RH and RT. After 7 days sample was analyzed by XRPD and the XRPD pattern is presented in FIG. 3A.

### Procedure C

LX9211 dihydrogen phosphate form S3 (1.9 g) was suspended in 10 ml of water for 1 day at RT. The suspension was then filtered off. Sample was dried at 40° C. for 6 h and analyzed with XRPD.

### Procedure D

Co-crystal of LX9211 dihydrogen phosphate: urea form U1 (10 mg) was dissolved in 0.2 mL 2-propanol: water mixture (1:2) at 35° C. Solution was left to evaporate. Obtained crystal was filtered off and analyzed by XRPD. LX9211 dihydrogen phosphate form S1 was obtained and the crystallographic data for the LX9211 dihydrogen phosphate form S1 are presented in

TABLE 1

| Space | | | | | |
|---|---|---|---|---|---|
| group | Cell lengths/Å | Cell angles | Cell volume | Z | T/K |
| I2 | a = 23.6683(4) | α = 90 | 4604.48 | 8 | 170 |
| | b = 7.80870(10) | β = 109.613(2) | | | |
| | c = 26.4480(5) | γ = 90 | | | |

### Procedure E

LX9211 (5 grams) was dissolved in 2-propanol (50 mL) (concentration 100 g/L) at room temperature (25° C.) to obtain a solution. Phosphoric acid (85%) was added dropwise (1.5 ml, 1.7 eq). Crystallization occurred after 1 hour. Obtained suspension was sampled, filtered off and analysed by XRPD. Mixture of LX9211 dihydrogen phosphate form S1 and S3 was obtained. More solvent (50 mL) was added in suspension mixture (suspension concentration 50 g/L). Suspension was stirred for additional 3 days and vacuum filtered. Sample was analysed by XRPD. LX9211 dihydrogen phosphate form S1 was obtained. Sample was dried in vacuum oven for 6 hours at 40° C. after which was analysed with XRPD. Again, LX9211 dihydrogen phosphate form S1 was obtained.

Form S1 shows a melting endotherm with an onset temperature of about 180-186° C.

### Example 4: Preparation of LX9211 Dihydrogen Phosphate Form S3

### Procedure A

LX9211 (1 gram) was dissolved in 2-propanol (10 mL) (concentration 100 g/L) at room temperature (25° C.) to obtain a solution. Phosphoric acid (85%) was added dropwise (0.284 ml, 1.6 eq). The obtained solution was left stirring at room temperature (25° C.) for 30 minutes. Crystallization occurred. Obtained solid was isolated by vacuum filtration. The solid was dried in a vacuum oven at 50° C. for 1 hour. The obtained solid was analyzed by XRD, characterized as LX9211 dihydrogen phosphate form S3 and the XRD pattern is presented in FIG. 4.

### Procedure B—Cyclic DSC Heating

LX9211 form S1 (3.5 mg) was subjected to thermal treatment on DSC Discovery TA instrument according to following steps:
1. Heating of the sample by heating rate 10° C./minute up to temperature of 85° C.,
2. Isothermal heating for 10 minutes at 85° C.,
3. Cooling to room temperature,
4. Heating of the sample by heating rate 10° C./minute up to temperature of 150° C.,
5. Isothermal heating for 5 minutes at 150° C.

Sample was cooled down to room temperature and analyzed by XRPD and identified as LX9211 dihydrogen phosphate form S3.

### Procedure C

LX9211 (300 mg) was dissolved in 2-propanol (1.2 mL) (concentration 250 g/L) at room temperature (25° C.) to obtain a solution. Phosphoric acid (85%) was added dropwise (0.1217 ml, 2.3 eq). The obtained solution was cooled in ice bath. The solution was left stirring for 3 days at room temperature. Crystallization occurred. Obtained solid was isolated by vacuum filtration, analyzed by XRD and identified as LX9211 dihydrogen phosphate form S3.

Form S3 shows a melting endotherm with an onset temperature of about 178-184° C.

### Example 5: Preparation of LX9211 Tosylate Form T1

LX9211 base (67.05 mg) was dissolved in 1 ml of ethanol/water mixture (1:1) at room temperature. P-toluene sulfonic acid monohydrate (2 eq. 32.95 mg) was added into solution. The solution was left under stirring at 0-10° C. for 30 minutes after which crystallization occurred. Suspension was filtrated off and analyzed by XRPD. The XRPD pattern is presented in FIG. 5.

### Example 6: Preparation of LX9211 Tosylate Form T2

LX9211 base (264.04 mg) was dissolved in 2.5 ml of 2-propanol/water mixture (2:1) at room temperature. P-toluene sulfonic acid monohydrate (2 eq. 235.96 mg) was added into solution. The solution was left under stirring at 0-10° C. after which crystallization occurred. Suspension was left to stir for 1 day at room temperature and then it was filtered off and analyzed by XRPD and the XRPD pattern is presented in FIG. 6.

### Example 7: Preparation of LX9211 Camsylate Form C1

LX9211 base (226.71 mg) was dissolved in 4 ml of acetone/water mixture (1:1) at room temperature. Camphor-10-sulfonic acid (2 eq. 273.29 mg) was added into solution. The solution was stirred at 0-10° C. after which crystallization occurred. Suspension was left to stir for 1 day at room temperature and then it was filtered off and analyzed by XRPD. LX9211 camsylate, form C1 was obtained (FIG. 7).

### Example 8: Preparation of LX9211 Camsylate Form C2

LX9211 camsylate form C1 (2 mg) was placed in a pin hole aluminum pan. Sample was subjected to thermal treatment in DSC Discovery TA instruments, according to following steps:
1. Heating of the sample by heating rate 10° C./minute up to temperature of 195° C.,
2. Isothermal heating for 15 minutes at 195° C.

Obtained solid was analyzed by XRPD. LX9211 camsylate, form C2 was obtained and the XRPD pattern is presented in FIG. 8.

### Example 9: Preparation of LX9211 Form S5 of LX9211: Phosphoric Acid

### Procedure A

LX9211 (300 mg) was dissolved in acetone/water 1:2 mixture (2 mL) (concentration 150/L) at room temperature (25° C.) to obtain solution. Phosphoric acid (85%) was added dropwise (23 μL, 0.4 eq) and solution was left to stir at room temperature. Crystallization occurred. Obtained solid was filtered and analyzed by XRPD. LX9211 form S5 was obtained and the XRPD pattern is presented in FIG. 9.

### Procedure B

LX9211 hemihydrogen phosphate form S5 (100 mg) was dissolved in 5 mL EtOH, 96% at room temperature for 3 weeks. After 3 weeks crystallization occurred. Obtained solid was filtered off and analyzed by XRPD. LX9211 hemihydrogen phosphate form S5 was obtained and the crystallographic data for the LX9211 hemihydrogen phosphate form S5 are presented in

TABLE 2

| Space | | | | | |
|---|---|---|---|---|---|
| group | Cell lengths/Å | Cell angles | Cell volume | Z | T/K |
| P1 | a = 7.4367(3) | α = 81.384(4) | 2158.05 | 1 | 170 |
| | b = 14.9363(8) | β = 85.922(4) | | | |
| | c = 19.8050(10) | γ = 83.568(4) | | | |

### Procedure C

400 mg of LX9211 base form A was dissolved in acetone (2 mL) (concentration 200 g/L) at room temperature (25° C.) to obtain solution. Phosphoric acid (85%) was added dropwise (60 μL, 0.9 eq) and solution was left to stir at room temperature. Crystallization occurred. Obtained solid was filtered and analyzed by XRPD. LX9211 form S5 was obtained. Form S5 shows a melting endotherm with an onset temperature of about 78-84° C.

### Example 10: Preparation of LX9211 Succinate Form J2

LX9211 (224.3 mg) was dissolved in 3 mL of acetone at room temperature (25° C.) to obtain solution. Succinic acid (75.6 mg) was added to solution and it was left to stir at room temperature. 3 mL of heptane added dropwise to solution after which crystallization occurred. Obtained solid was filtered and analyzed by XRPD. LX9211 succinate form J2 was obtained and XRPD pattern is presented in FIG. 10.

### Example 11: Preparation of LX9211 Fumarate Form F3

LX9211 base (225.3 mg, 1 eq) and fumaric acid (74.7 mg, 1.1 eq) were dissolved in 3 mL of 2-Propanol at 35° C. to obtain solution. Heating was discontinued and solution was cooled down to RT. After 30 minutes crystallization occurred. Obtained solid was filtered and analyzed by XRPD. LX9211 fumarate form F3 was obtained and the XRPD pattern is presented in FIG. 11.

### Example 12: Preparation of LX9211 (−)-Camsylate Form K1

LX9211 base (360.8 mg) was dissolved in 5 ml of 2-propanol at room temperature. (−)-10-camphorsulfonic acid (1.1 eq.; 239.2 mg) was added into solution. The solution was left to stir at RT for 3 hours. After crystallization occurred, the suspension was filtrated off and the obtained material analyzed by XRPD. LX9211 camsylate, form K1 was obtained and the XRPD pattern is presented in FIG. 12.

Form K1 shows a melting endotherm with an onset temperature of about 218-222° C.

### Example 13: Preparation of LX9211 Citrate Form L1

LX9211 base (1500 mg) was dissolved in 17 ml of acetonitrile at room temperature. Citric acid (1.1 eq.; 823 mg) was added into solution. The solution was left to stir at RT for 2 hours after which crystallization occurred. The suspension was filtrated off and analyzed by XRPD. LX9211 citrate, form L1 was obtained and the XRPD pattern is presented in FIG. 13.

### Example 14: Preparation of LX9211 Citrate Form L2

LX9211 citrate form L1 (200 mg) was suspended in 2 ml of water at room temperature. Suspension was left to stir for 1 day and then it was filtrated off, dried in a vacuum oven at 40° C. for 2 hours and obtained material was analyzed by XRPD. LX9211 citrate, form L2 was obtained and the XRPD pattern is presented in FIG. 14.

### Example 15: Preparation of LX9211 Oxalate Form O5

LX9211 base (751 mg, 1 eq) and Oxalic acid (249 mg, 1.1 eq) were dissolved in 16 ml of mixture of solvents acetone: water (1:10) at 60° C. Heating was discontinued and the reaction mixture was spontaneously cooled down do RT. The obtained suspension was left to stir for 1 day then filtrated off. Sample was dried in a vacuum oven at 50° C. for 1.5 hour and the obtained material was analyzed by XRPD. LX9211 oxalate form O5 was obtained and the XRPD pattern is presented in FIG. 15.

### Example 16: Preparation of LX9211 Tartrate Form V2

LX9211 base (35 mg, 1 eq) and L-(+)-Tartaric acid (15 mg, 1.1 eq) were stirred in 0.5 ml of acetonitrile at room temperature for 2 days. The suspension was filtrated off and the obtained material was analyzed by XRPD. LX9211 tartrate form V2 was obtained and the XRPD pattern is presented in FIG. 16.

### Example 17: Preparation of LX9211 Dihydrogen Phosphate: Urea Form U1

### Procedure A

LX9211 phosphate form S3 (176 mg, 1 eq) and urea (40 mg, 2 eq) were stirred in 6 ml of anhydrous acetonitrile at 60° C. for 2 hours. Suspension was spontaneously cooled down to room temperature and stirred for 3 days. The suspension was filtrated off, and the obtained material was dried in vacuum oven at 50° C. for 3.5 hours and analyzed by XRPD. LX9211 dihydrogen phosphate: Urea form U1 was obtained and the XRPD pattern is presented in FIG. 17.

Form U1 shows a melting endotherm with an onset temperature of about 163-167° C.

### Procedure B

Co-crystal of LX9211 dihydrogen phosphate: urea form U1 (10 mg) was dissolved in 0.2 mL of Methyl isobutyl ketone at 85° C. Solution was left to evaporate. Obtained powder was filtered off and analyzed by XRPD. LX9211 dihydrogen phosphate: Urea form U1 was obtained.

### Procedure C

Co-crystal of LX9211 dihydrogen phosphate: urea form U1 (10 mg) was dissolved in 0.6 mL of tetrahydrofuran at 55° C. Solution was left to evaporate. Obtained powder was filtered off and analyzed by XRPD. LX9211 dihydrogen phosphate: Urea form U1 was obtained.

### Example 18: Preparation of LX9211 Dihydrogen Phosphate: L-(+)-Tartaric Acid Form V3

LX9211 phosphate form S1 (150 mg, 1 eq, obtained according to Example 3, procedure C) and L-(+)-Tartaric acid (50 mg, 1.1 eq) were stirred in 2 ml of acetonitrile at room temperature for 1 day. The suspension was filtrated off and the obtained material was analyzed by XRPD. LX9211 dihydrogen phosphate: L-(+)-tartaric acid form V3 was obtained and the XRPD pattern is presented in FIG. 18.

### Example 19: Preparation of LX9211 Dihydrogen Phosphate: Oxalic Acid Form O4

LX9211 phosphate form S5 (45 mg, 1 eq) and Oxalic acid (5 mg, 1.1 eq) were stirred in 0.5 ml of acetonitrile at room temperature for 3 days. The suspension was filtrated off and the obtained material was analyzed by XRPD. LX9211 dihydrogen phosphate: Oxalic acid form O4 was obtained and the XRPD pattern is presented in FIG. 19.

### Example 20: Preparation of LX9211 Form C

LX9211 base form A (80 mg) and 8 mL of was water was added to a vial and stirred in chamber at 37° C. for 24 hours. After 24 hours the sample was spontaneously cooled down to RT and was filtered off. The obtained material was analyzed by XRPD. LX9211 base form C was obtained and the XRPD pattern is presented in FIG. 20.

Form C shows a melting endotherm with an onset temperature of about 41-43° C.

### Example 21: Preparation of LX9211 Dihydrogen Phosphate Form S4

LX9211 base form A (200 mg) was dissolved in 2 ml of 2-propanol/water (ratio 3:2) mixture at room temperature. 32 μL of phosphoric acid (eq. 1.1) was added in solution. Solution was left to stir at RT for 1 hour when crystallization occurred. Obtained solid was filtered off and analyzed by XRPD. LX9211 phosphate form S4 was obtained and the XRPD pattern is presented in FIG. 21.

### Example 22: Preparation of LX9211 Oxalate Form O2

About 15 mg of LX9211 oxalate form O5 was dissolved in 0.2 ml of 2-propanol/water mixture (ratio 1:1) at 80° C. Obtained solution was left to cool down at RT for 1 day after which crystallization occurred. Obtained solid was filtered off and analyzed by XRPD. LX9211 oxalate form O2 was obtained and the XRPD pattern is presented in FIG. 22.

### Example 23: Preparation of LX9211 L-(+)-Tartrate Form V5

1.05 grams of LX9211 base (1 eq) and 0.45 g of L-(+)-Tartaric acid were dissolved in 6 mL of 2-Propanol at 60° C. After 15 minutes, crystallization occurred. Obtained suspension was left to cool down at RT, stirred for 2 hours, filtered off and analyzed by XRPD. LX9211 tartrate form V5 was obtained and the XRPD pattern is presented in FIG. 23.

### Example 24: Stability Studies

### Form S1 LX9211 Dihydrogen Phosphate

Samples of LX9211 dihydrogen phosphate form S1 were subjected to conditions of different relative humidities (40%-100%) at ambient temperature. XRPD analysis was performed on the samples after 30 days. The results are shown in Table 3 below:

TABLE 3
Relative humidity (30 days)

| 40% | 60% | 80% | 100% |
|---|---|---|---|
| No | No | No | No |
| polymorphic | polymorphic | polymorphic | polymorphic |
| conversion | conversion | conversion | conversion |

Samples of LX9211 dihydrogen phosphate form S1 were subjected to strong grinding and to solvent drop grinding in acetone, ethanol (96%), and 2-propanol). Grinding was carried out on the samples alone, or in the presence of the solvent. In these experiments, about 20 mg of the sample is placed in a mortar and ground with a pestle for 2 minutes. The solvent, when used, as added to the crystalline material before grinding, in a volume of 10 microliters. XRPD analysis performed on each of the samples after the grinding experiment, confirmed no change in the starting material (Table 4):

TABLE 4

| Condition | XRPD analysis results |
|---|---|
| No solvent | No polymorphic conversion |
| Acetone | No polymorphic conversion |
| Ethanol (96%) | No polymorphic conversion |
| 2-propanol | No polymorphic conversion |

A sample of LX9211 dihydrogen phosphate form SI was subjected to a pressure of 1 ton (Perkin Elmer Hydraulic Press, set to 1 tons). XRPD analysis was performed on the samples after 2 minutes showed no polymorphic conversion.

The above results demonstrate that Form S1 of LX9211 dihydrogen phosphate has good storage stability, is resistant to polymorphic changes during grinding or compression, and is therefore highly suitable for preparing pharmaceutical formulations.

### Form S3 LX9211 Dihydrogen Phosphate

Samples of LX9211 dihydrogen phosphate form S3 were subjected to conditions of different relative humidities (20%-80%) at ambient temperature. XRPD analysis was performed on the samples after 30 days. The results are shown in Table 5 below:

TABLE 5
Relative humidity (30 days)

| 20% | 40% | 60% | 80% |
|---|---|---|---|
| No | No | No | No |
| polymorphic | polymorphic | polymorphic | polymorphic |
| conversion | conversion | conversion | conversion |

Samples of LX9211 dihydrogen phosphate form S3 were subjected to strong grinding and to solvent drop grinding in acetone, ethanol (96%), and 2-propanol). Grinding was carried out on the samples alone, or in the presence of the solvent. In these experiments, about 20 mg of the sample is placed in a mortar and ground with a pestle for 2 minutes. The solvent, when used, as added to the crystalline material before grinding, in a volume of 10 microliters. XRPD analysis performed on each of the samples after the grinding experiment, confirmed no change in the starting material (Table 6):

TABLE 6

| Condition | XRPD analysis results |
|---|---|
| No solvent | No polymorphic conversion |
| Acetone | No polymorphic conversion |
| Ethanol (96%) | No polymorphic conversion |
| 2-propanol | No polymorphic conversion |

A sample of LX9211 dihydrogen phosphate form S3 was subjected to a pressure of 1 ton (Perkin Elmer Hydraulic Press, set to 1 tons). XRPD analysis was performed on the samples after 2 minutes showed no polymorphic conversion.

The above results demonstrate that Form S3 of LX9211 dihydrogen phosphate has good storage stability, is resistant to polymorphic changes during grinding or compression, and is therefore especially suitable for preparing pharmaceutical formulations.

Form K1 LX9211 camsylate

Samples of LX9211 camsylate form K1 were subjected to conditions of different relative humidities (20%-100%) at ambient temperature. XRPD analysis was performed on the samples after 30 days. The results are shown in Table 7:

TABLE 7
Relative humidity (30 days)

| 20% | 40% | 60% | 80% | 100% |
|---|---|---|---|---|
| No | No | No | No | No |
| polymorphic | polymorphic | polymorphic | polymorphic | polymorphic |
| conversion | conversion | conversion | conversion | conversion |

Moreover, DVS studies showed less than 0.1% water uptake at least up to 90% RH, indicating that Form K1 of LX9211 camsylate is non-hygroscopic.

Samples of LX9211 camsylate form K1 were subjected to strong grinding and to solvent drop grinding in acetone, ethanol (96%), and 2-propanol). Grinding was carried out on the samples alone, or in the presence of the solvent. In these experiments, about 20 mg of the sample is placed in a mortar and ground with a pestle for 2 minutes. The solvent, when used, as added to the crystalline material before grinding, in a volume of 10 microliters. XRPD analysis performed on each of the samples after the grinding experiment, confirmed no change in the starting material (Table 8):

TABLE 8

| Condition | XRPD analysis results |
|---|---|
| No solvent | No polymorphic conversion |
| Acetone | No polymorphic conversion |
| Ethanol (96%) | No polymorphic conversion |
| 2-propanol | No polymorphic conversion |

A sample of LX9211 camsylate form K1 was subjected to a pressure of 1 ton (Perkin Elmer Hydraulic Press, set to 1 tons). XRPD analysis was performed on the samples after 2 minutes showed no polymorphic conversion.

These results show that LX9211 camsylate form K1 has good storage stability, is resistant to polymorphic changes during grinding or compression, and is therefore highly suitable for preparing pharmaceutical formulations.

### Form S5 LX9211 Hemihydrogen Phosphate

Samples of LX9211 hemihydrogen phosphate form S5 were subjected to conditions of different relative humidities (20%-100%) at ambient temperature. XRPD analysis was performed on the samples after 30 days. The results are shown in Table 9:

TABLE 9
Relative humidity (30 days)

| 20% | 40% | 60% | 80% | 100% |
|---|---|---|---|---|
| No | No | No | No | No |
| polymorphic | polymorphic | polymorphic | polymorphic | polymorphic |
| conversion | conversion | conversion | conversion | conversion |

Samples of LX9211 hemihydrogen phosphate form S5 were subjected to strong grinding and to solvent drop grinding in acetone, ethanol (96%), and 2-propanol). Grinding was carried out on the samples alone, or in the presence of the solvent. In these experiments, about 20 mg of the sample is placed in a mortar and ground with a pestle for 2 minutes. The solvent, when used, as added to the crystalline material before grinding, in a volume of 10 microliters. XRPD analysis performed on each of the samples after the grinding experiment, confirmed no change in the starting material (Table 10):

TABLE 10

| Condition | XRPD analysis results |
|---|---|
| No solvent | No polymorphic conversion |
| Acetone | No polymorphic conversion |
| Ethanol (96%) | No polymorphic conversion |
| 2-propanol | No polymorphic conversion |

A sample of LX9211 hemihydrogen phosphate form S5 was subjected to a pressure of 1 ton (Perkin Elmer Hydraulic Press, set to 1 tons). XRPD analysis was performed on the samples after 2 minutes showed no polymorphic conversion.

The above data demonstrate that Form S5 of LX9211 hemihydrogen phosphate has good storage stability, is resistant to polymorphic changes during grinding or compression, and is therefore especially suitable for preparing pharmaceutical formulations.

### LX9211 Dihydrogen Phosphate and Urea Form U1

The solubilities of LX9211 dihydrogen phosphate forms S1 and S3 and LX9211 dihydrogen phosphate and urea, and LX9211 free base form A were determined in water and pH 4.5 buffer as follows:

About 80 mg of samples of the starting materials were weighed into 15 ml PP centrifuge tubes. 8 ml of appropriate buffer solution (pH 4.5 according to USP 35-NF 30 Buffer Solutions) or water was added. Tubes were stoppered and shaken at 160 rpm for 24 hours at 37±0.2° C. in an incubator shaker (Innova 4080, New Brunswick Scientific). Suspensions were filtered through 0.45 μm cellulose syringe filter (CHROMAFIL PTFE-45/25, Macherey-Nagel). Filtrates were sampled and concentration of the starting material was determined by HPLC according to corresponding standard. The results are shown in Table 11 below:

TABLE 11

Solubility (mg/mL)

| LX9211 form | pH 4.5 | water |
|---|---|---|
| LX9211 dihydrogen phosphate | 8.1 | 25.0 |
| form S1 | | |
| LX9211 dihydrogen phosphate | 13.8 | 25.0 |
| form S3 | | |
| LX9211 dihydrogen phosphate and urea | 16.4 | 24.4 |
| form U1 | | |
| LX9211 free base form A | 9.7 | 0.1 |

The results demonstrate the excellent superior solubility the LX9211 forms according to the disclosure.

1. A crystalline form of LX9211 dihydrogen phosphate designated form S1, which is characterized by dada selected from the group consisting of:
(a) an X-ray powder diffraction pattern having peaks at 7.0, 14.0 and 16.7 degrees 2-theta±0.2 degrees 2-theta;
(b) an X-ray powder diffraction pattern substantially as depicted in FIG. 3 or 3A;
(c) a solid state ¹³C NMR spectrum having characteristic peaks at 152.4, 120.8, 56.0 and 24.2 ppm±0.2 ppm;
(d) a solid state ¹³C NMR spectrum having the following chemical shift absolute differences from reference peak at 20.1 ppm±0.2 ppm: 132.3, 100.7, 35.9 and 4.1 ppm±0.1 ppm;
(e) a solid state ¹³C NMR spectrum substantially as depicted in FIG. 24;
(f) the following unit cell data:

| | |
|---|---|
| cell length a | 23.67 Å |
| cell length b | 7.81 Å |
| cell length c | 26.45 Å |
| cell angle alpha | 90° |
| cell angle beta | 109.6° |
| cell angle gamma | 90° |
| symmetry space group name | I2; and |

(g) a combination of any two or more of (a)-(f).

2. The crystalline form of LX9211 dihydrogen phosphate according to claim 1, which is characterized by an X-ray powder diffraction pattern having peaks at 7.0, 14.0 and 16.7 degrees 2-theta±0.2 degrees 2-theta, and also having any one or both additional peaks selected from 20.2 and 21.9 degrees 2-theta±0.2 degrees 2-theta.

3. The crystalline form of LX9211 dihydrogen phosphate according to claim 1, which is characterized by an X-ray powder diffraction pattern having peaks at 7.0, 14.0, 16.7, 20.2 and 21.9 degrees 2-theta±0.2 degrees 2-theta.

4. The crystalline form of LX9211 dihydrogen phosphate according to claim 1, which is characterized by an X-ray powder diffraction pattern having peaks at 7.0, 14.0, 16.7, 20.2 and 21.9 degrees 2-theta±0.2 degrees 2-theta, and also having any one, two, three, four or five additional peaks selected from 4.4, 8.7, 10.0, 17.5 and 18.2 degrees 2-theta±0.2 degrees 2-theta.

5. The crystalline form of LX9211 dihydrogen phosphate according to claim 1, which is characterized by an X-ray powder diffraction pattern having peaks at 4.4, 7.0, 8.7, 10.0, 14.0, 16.7, 17.5, 18.2, 20.2 and 21.9 degrees 2-theta±0.2 degrees 2-theta.

6. The crystalline form of LX9211 dihydrogen phosphate according to claim 1, wherein the crystalline form is a hydrate.

7. (canceled)

8. (canceled)

9. (canceled)

10. (canceled)

11. (canceled)

12. (canceled)

13. A crystalline form of LX9211 hemi-hydrogen phosphate designated form S5, which is characterized by data selected from the group consisting of:
(a) an X-ray powder diffraction pattern having peaks at 4.5, 11.9 and 18.1 degrees 2-theta±0.2 degrees 2-theta;
(b) an X-ray powder diffraction pattern substantially as depicted in FIG. 9;
(c) a solid state ¹³C NMR spectrum having characteristic peaks at 150.4, 123.0, 113.9 and 26.6 ppm±0.2 ppm;
(d) a solid state ¹³C NMR spectrum having the following chemical shift absolute differences from reference peak at 21.4 ppm±0.2 ppm: 129.0, 101.6, 92.5 and 5.2 ppm±0.1 ppm;
(e) a solid state ¹³C NMR spectrum substantially as depicted in FIG. 26;
(f) the following unit cell data:

| | |
|---|---|
| cell length a | 7.44 Å |
| cell length b | 14.94 Å |
| cell length c | 19.81 Å |
| cell angle alpha | 81.4° |
| cell angle beta | 85.9° |
| cell angle gamma | 83.6° |
| symmetry space group name | P1; and |

(g) a combination of any two or more of (a)-(f).

14. The crystalline form of LX9211 hemi-hydrogen phosphate according to claim 13, which is characterized by an X-ray powder diffraction pattern having peaks at 4.5, 11.9 and 18.1 degrees 2-theta±0.2 degrees 2-theta, and also having any one or both additional peaks selected from 6.9 and 10.0 degrees 2-theta±0.2 degrees 2-theta.

15. The crystalline form of LX9211 hemi-hydrogen phosphate according to claim 13, which is characterized by an X-ray powder diffraction pattern having peaks at 4.5, 6.9, 10.0, 11.9 and 18.1 degrees 2-theta±0.2 degrees 2-theta.

16. The crystalline form of LX9211 hemi-hydrogen phosphate according to claim 13, which is characterized by an X-ray powder diffraction pattern having peaks at 4.5, 6.9, 10.0, 11.9 and 18.1 degrees 2-theta±0.2 degrees 2-theta, and also having any one, two, three, four or five additional peaks selected from 12.5, 17.7, 20.1, 25.7 and 27.8 degrees 2-theta±0.2 degrees 2-theta.

17. The crystalline form of LX9211 hemi-hydrogen phosphate according to claim 13, which is characterized by an X-ray powder diffraction pattern having peaks at 4.5, 6.9, 10.0, 11.9, 12.5, 17.7, 18.1, 20.1, 25.7 and 27.8 degrees 2-theta±0.2 degrees 2-theta.

18. The crystalline product form of LX9211 hemi-hydrogen phosphate according to claim 13, wherein the crystalline form is a hydrate.

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. The crystalline form of LX9211 dihydrogen phosphate according to claim 1, which is isolated.

32. The crystalline form of LX9211 dihydrogen phosphate according to claim 1, which is polymorphically pure; wherein the crystalline form contains: about 20% (w/w) or less, about 10% (w/w) or less, about 5% (w/w) or less, about 2% (w/w) or less, about 1% (w/w) or less, or about 0% of any other crystalline forms of the same compound, and/or wherein the crystalline form contains: about 20% (w/w) or less, about 10% (w/w) or less, about 5% (w/w) or less, about 2% (w/w) or less, about 1% (w/w) or less, or about 0% of any amorphous forms of the same compound.

33. (canceled)

34. (canceled)

35. A pharmaceutical composition or formulation comprising a crystalline form according to claim 1, and at least one pharmaceutically acceptable excipient, optionally in the form of a solid dosage form, as a capsule or tablet.

36. A process for preparing a pharmaceutical composition or formulation comprising a crystalline form according to claim 1, and at least one pharmaceutically acceptable excipient, optionally in the form of a solid dosage form, the process comprising combining the crystalline form according to claim 1 with at least one pharmaceutically acceptable excipient.

37. (canceled)

38. (canceled)

39. A method of treating Diabetic Peripheral Neuropathic pain or Post-Herpetic Neuralgia, comprising administering a therapeutically effective amount of any one or a combination of crystalline form according to claim 1 to a subject in need of treatment.

40. A pharmaceutical composition or formulation comprising the crystalline form according to claim 13, and at least one pharmaceutically acceptable excipient, optionally in the form of a solid dosage form, as a capsule or tablet.

41. A process for preparing a pharmaceutical composition or formulation comprising the crystalline form according to claim 13, and at least one pharmaceutically acceptable excipient, the process comprising combining the crystalline form according to claim 13 with at least one pharmaceutically acceptable excipient.

42. A method of treating Diabetic Peripheral Neuropathic pain or Post-Herpetic Neuralgia, comprising administering a therapeutically effective amount of any one or a combination of the crystalline form according to claim 13 to a subject in need of treatment.

43. The crystalline form of LX9211 hemi-hydrogen phosphate according to claim 13, which is isolated.

44. The crystalline form of LX9211 hemi-hydrogen phosphate according to claim 13, which is polymorphically pure; wherein the crystalline form contains: about 20% (w/w) or less, about 10% (w/w) or less, about 5% (w/w) or less, about 2% (w/w) or less, about 1% (w/w) or less, or about 0% of any other crystalline forms of the same compound, and/or wherein the crystalline form contains: about 20% (w/w) or less, about 10% (w/w) or less, about 5% (w/w) or less, about 2% (w/w) or less, about 1% (w/w) or less, or about 0% of any amorphous forms of the same compound.
